(19) United States
(12) Patent Application Publication
(10) Pub. No.: US 2025/0205257 A1
(43) Pub. Date: Jun. 26, 2025

(54) CYCLIC CIDOFOVIR OR A PRODRUG THEREOF FOR USE IN THE TREATMENT OR PREVENTION OF AFRICAN SWINE FEVER

(71) Applicant: VIROVET NV, Heverlee (BE)

(72) Inventors: Eleonora KISS, Heverlee (BE); Robert VRANCKEN, Berchem (BE); Jérôme VILLERS, Mellet (BE); Nesya GORIS, Schoten (BE); Linda DIXON, Guildford, Surrey (GB); Leah GOULDING, Ottery St. Mary, Devon (GB)

(73) Assignee: VIROVET NV, Heverlee (BE)

(21) Appl. No.: 18/850,745

(22) PCT Filed: May 5, 2023
(86) PCT No.: PCT/EP2023/061947

§ 371 Date: Sep. 25, 2024

(30) Foreign Application Priority Data
May 6, 2022 (EP) 22172077.4

(51) Int. Cl. A61K 31/675; A61P 31/20

(52) U.S. Cl. CPC A61K 31/675; A61P 31/20

(57) ABSTRACT

Provided herein is cyclic cidofovir for use in the treatment of African Swine Fever (ASF) in an animal comprising administering orally in a dose of from 10.0 to. 30.0 mg/kg of said cyclic cidofovir, wherein at least one dose of cyclic cidofovir, such as one or two doses, is administered to the animal daily for a first period of from 1 to 4 consecutive days and one dose of cyclic cidofovir is administered to the animal every other day or daily for a second period of from 5 to 14 consecutive days, and wherein the average daily dose administered in said first period is higher than the average daily dose administered in said second period.

### FIELD OF THE INVENTION

The present invention relates to methods for treating or preventing African swine fever and to compositions for use therein.

### BACKGROUND OF THE INVENTION

Forty percent of the world's meat consumption is pork, with Europe being the second largest pork producer in the world with approximately 150 million pigs. However, as industrial systems are characterized by large numbers of animals being raised, predominantly in confinement, with rapid population turnover at a single site, the meat industry is vulnerable to viral disease outbreaks, which can lead to huge financial losses, threats to food security and animal suffering.

During the past decade, African swine fever (ASF) has spread from the Caucasus region to European countries affecting domestic pig and wild boar populations. ASF is a highly infectious and deadly disease of pigs. More particularly, ASF in domestic pigs has a mortality rate up to 100% and is characterized by high fever, depression, loss of appetite, vomiting, and hemorrhages of skin and internal organs.

At present, there is no effective vaccine or treatment available for ASF and introduction and spread of ASF onto domestic pig farms can only be prevented by strict compliance to control measures. Important control measures include the identification of animals and farm records, the containment of pigs, so as to not allow direct or indirect pig-pig and/or pig-wild boar contacts, and proper disposal of manure and dead animals. As a result thereof, ASF poses a serious constraint to the development of both smallholder and industrial pig industries.

Thus, there is a need for the development of effective means for preventing spread of ASF.

### SUMMARY OF THE INVENTION

Present inventors were to their knowledge the first to demonstrate the in vivo efficacy of a nucleoside phosphonate, and more particularly cyclic cidofovir, in the treatment (defined herein as to encompass treatment and prevention of the disease) of African Swine Fever virus (ASFV) infected animals, and more particularly pigs. Furthermore, present inventors have tested several dosage regimes and have found a particular dose range as well as a particular dosage regime for cyclic cidofovir or a prodrug thereof allowing treatment (including prevention of disease) of ASFV infected animals, and more particularly pigs. More particularly, present inventors found a dosage regime comprising two subsequent periods wherein the dose and/or timing of administration in the first period is different from the dose and/or timing in the second period, preferably wherein the average daily dose administered in the first period is higher than the average daily dose administered in the second period, reduced viral shedding and clinical signs of disease and improved survival in pigs challenged with ASFV, while allowing an acceptable or absent toxicity of cyclic cidofovir or a prodrug.

Accordingly, a first aspect provides a cyclic cidofovir for use in the treatment of African Swine Fever (ASF) in an animal comprising administering orally in a dose of from 10.0 to. 30.0 mg/kg of said cyclic cidofovir, wherein at least one dose of cyclic cidofovir, such as one or two doses, is administered to the animal daily for a first period of from 1 to 4 consecutive days and one dose of cyclic cidofovir is administered to the animal every other day or daily for a second period of from 5 to 14 consecutive days, and wherein the average daily dose administered in said first period is higher than the average daily dose administered in said second period. In particular embodiments, the cyclic cidofovir for use envisaged herein is a compound of formula (i):

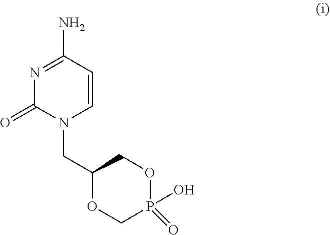

or an ester, an amidate or esteramidate thereof, more particularly an acyloxyalkyl ester, alkoxycarbonyloxyalkyl ester, alkoxyalkyl ester, or a phosphoramidate or phosphonamidite thereof.

In some embodiments the cyclic cidofovir for use according to the present invention has a structure of formula (I) or (II),

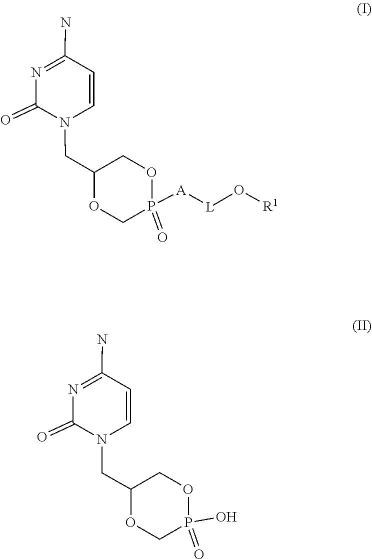

wherein,
A is selected from the group consisting of O and NH;
L is selected from the group consisting of —(CR²R³)ₙ—, —CH(R⁴)—C(O)— and —C(O)—CH(R⁵)—;
n is an integer selected from 1, 2, 3, 4 or 5;
R¹ is selected from the group consisting of C₆₋₂₅alkyl, —C(O)R⁶, —CO₂R⁷, C₆₋₂₅haloalkyl, C₆₋₂₅alkenyl, C₆₋₂₅haloalkenyl, C₃₋₁₂cycloalkyl and C₃₋₁₂halocycloalkyl;
each R² is independently selected from the group consisting of hydrogen, C₁₋₆alkyl, C₃₋₁₂cycloalkyl and halogen;
each R³ is independently selected from the group consisting of hydrogen, C₁₋₆alkyl, C₃₋₁₂cycloalkyl and halogen;
each R⁴ is independently selected from the group consisting of hydrogen, C₁₋₆alkyl, C₃₋₁₂cycloalkyl and halogen;
each R⁵ is independently selected from the group consisting of hydrogen, C₁₋₆alkyl, C₃₋₁₂cycloalkyl and halogen;
each R⁶ is independently selected from the group consisting of C₁₋₁₀alkyl, C₁₋₁₀haloalkyl, C₃₋₁₂cycloalkyl, C₃₋₁₂halocycloalkyl, C₂₋₁₀alkeny, and C₂₋₁₀haloalkenyl;
each R⁷ is independently selected from the group consisting of C₁₋₁₀alkyl, C₁₋₁₂haloalkyl, C₃₋₁₂cycloalkyl, C₃₋₁₂halocycloalkyl, C₂₋₁₀alkeny, and C₂₋₁₀haloalkenyl;
or a stereoisomer, tautomer, solvate, hydrate, pharmaceutically acceptable salt thereof.

In some embodiments the cyclic cidofovir for use according to the present invention has a structure of formula (I), wherein L is selected from the group consisting of —(CR²R³)ₙ— and —CH(R⁴)—C(O)—;
n is an integer selected from 1, 2, or 3;
R¹ is selected from the group consisting of C₆₋₂₅alkyl, —C(O)R⁶, —CO₂R⁷, C₆₋₂₅haloalkyl, C₆₋₂₅alkenyl, and C₃₋₁₀cycloalkyl;
each R² is independently selected from the group consisting of hydrogen, C₁₋₆alkyl, and halogen;
each R³ is independently selected from the group consisting of hydrogen, C₁₋₆alkyl, and halogen;
each R⁴ is independently selected from the group consisting of hydrogen, C₁₋₆alkyl, and halogen;
each R⁶ is independently selected from the group consisting of hydrogen, C₁₋₆alkyl, C₃₋₁₀cycloalkyl and halogen;
each R⁶ is independently selected from the group consisting of C₁₋₆alkyl, C₁₋₆haloalkyl, and C₃₋₆cycloalkyl;
each R⁷ is independently selected from the group consisting of C₁₋₆alkyl, C₁₋₆haloalkyl, and C₃₋₆cycloalkyl.

In some embodiments the cyclic cidofovir for use according to the present invention is selected from the group consisting of:

| ID | Structure |
|---|---|
| cHPMPC | 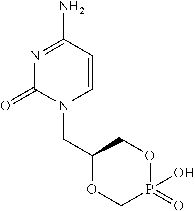 |
| 1 | 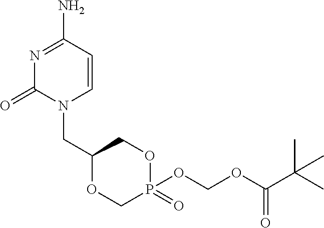 |
| 2 | 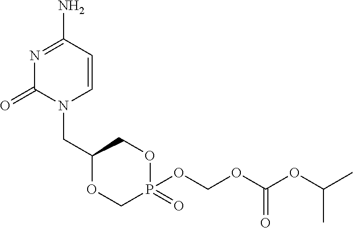 |
| 3 | 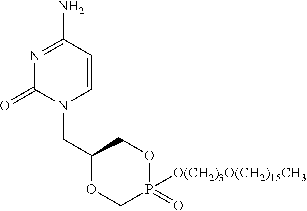 |
| 4 | 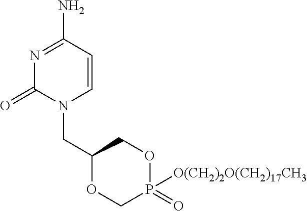 |
| 5 | 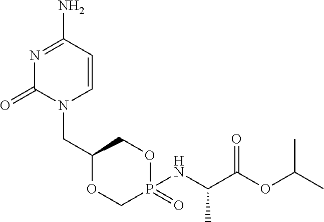 |

In particular embodiments, two doses of cyclic cidofovir are administered to the animal daily for a first period of 3 consecutive days and one dose of cyclic cidofovir is administered to the animal daily for a second period of from 5 to 14 consecutive days, preferably from 5 to 7 consecutive days.

In particular embodiments, one dose of cyclic cidofovir is administered to the animal daily for a first period of 3 consecutive days and one dose of cyclic cidofovir is administered to the animal every other day for a second period of from 5 to 14 consecutive days, preferably from 5 to 7 consecutive days.

In particular embodiments, the cyclic cidofovir is comprised in a pharmaceutical composition, preferably wherein said pharmaceutical composition is an aqueous solution.

In particular embodiments, the animal is a pig.

In particular embodiments, the animal is housed in a ASF surveillance or protection zone.

In particular embodiments, the administration of said cyclic cidofovir prevents the spread of ASF in an animal population.

### BRIEF DESCRIPTION OF DRAWINGS

FIG. 1. Summary outline of the VV03-0035 challenge study in pigs as described in Example 5. Critical study time points and main study results are depicted.

FIG. 2. Survival curves are shown for control and treated pigs of the VV03-0035 challenge study as described in Example 5. The x-axis shows the day post challenge with ASFV. The y-axis shows the percentage of pigs that survived beyond the moderate severity humane endpoint on each day. The median survival was calculated as 6 days for the control group and 19.5 days for the treated group.

FIG. 3. ASFV genome copy numbers in whole blood isolated from control pigs (C1 to C3) and treated pigs (T4 to T9). The viral genome copies were measured by quantitative qPCR and expressed as log₁₀ genome copies per ml of blood.

FIG. 4. ASFV genome copy numbers in nasal, oral and rectal swab samples collected from control pigs (C1 to C3) and treated pigs (T4 to T9). The viral genome copies were measured by quantitative qPCR and expressed as log₁₀ genome copies per ml.

FIG. 5. Survival curves are shown for control and treated pigs of the VV03-0035 challenge study as described in Example 6. The x-axis shows the day post challenge with ASFV. The y-axis shows the percentage of pigs that survived beyond the moderate severity humane endpoint on each day. The median survival was calculated as 5.5 days for the control group and 8 days for the treated group. This difference was statistically significant as determined by log-rank (Mantel-Cox) test and Gehan-Breslow-Wilcoxon test with p-values of 0.0010 and 0.0016, respectively.

FIG. 6. Results of tissue samples collected at necropsy including spleen, lung, sub-mandibular (SMLN), renal (RNL) and gastro-hepatic lymph (GHLN) nodes. Results for individual pigs in control or treatment group are shown on different columns and each panel compares results from tissues as indicated. The significance was determined by nested t-test, comparing control to treated pigs (*p 0.05, *** p 0.001).

FIG. 7. ASFV genome copy numbers in whole blood isolated from control pigs (C1 to C6) and treated pigs (T1 to T6). The viral genome copies were measured by quantitative qPCR and expressed as log₁₀ genome copies per ml of blood. The significance was determined by nested t-test, comparing control to treated pigs (* p 0.05, *** p 0.001).

FIG. 8. Shows photographs of microplates wherein expression of Green fluorescent protein by porcine bone marrow cells infected with a recombinant ASFV genotype II Georgia virus, GeorgiaDMGFB, expressing mNeon Green fluorescent protein, can be observed. Section A shows the results (i.e., expression of Green fluorescent protein) when ASFV-infected porcine bone marrow cells were exposed to no compound (columns 1, 2 and 3), Compound 3 (columns 4, 5 and 6), Compound 4 (columns 7, 8 and 9) or cHPMPC (columns 10, 11 and 12). The final concentrations of compounds tested were: A: 1.0 μM, B: 0.5 μM, C: 0.1 μM, D: 0.05 μM, E: 0.01 μM, F: 0.005 μM, G 0.001 μM. Section B shows the results when ASFV-infected porcine bone marrow cells were exposed to no compound (columns 1, 2 and 3), Compound 1 (columns 4, 5 and 6), Compound 2 (columns 7, 8 and 9) or Compound 5 (columns 10, 11 and 12). The final concentrations of compounds tested were: A: 1.0 μM, B: 0.5 μM, C: 0.1 μM, D: 0.05 μM, E: 0.01 μM, F: 0.005 μM, G 0.001 μM.

### DETAILED DESCRIPTION OF THE INVENTION

The term “about”, when used in relation to a numerical value, has the meaning generally understood in the relevant art. In certain embodiments the term “about” may be left out or may be interpreted to mean the numerical value +10%; or +5%; or +2%; or +1%.

Whenever used herein in relation to a percentage, w/w means weight/weight and w/v means weight/volume.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” when referring to recited members, elements or method steps also include embodiments which “consist of” said recited members, elements or method steps.

Furthermore, the terms first, second, third and the like in the description and in the claims, are used for distinguishing between similar elements or steps and not necessarily for describing a sequential or chronological order, unless specified. It is to be understood that the terms so used are interchangeable under appropriate circumstances and that the embodiments described herein are capable of operation in other sequences than described or illustrated herein.

Reference throughout this specification to “one embodiment” or “an embodiment” means that a particular feature, structure or characteristic described in connection with the embodiment is included in at least one embodiment envisaged herein. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

Any references cited herein are hereby incorporated by reference.

Present inventors were to their knowledge the first to demonstrate the in vivo efficacy of a nucleoside phosphonate in the treatment (defined herein as to encompass treatment and prevention) of African Swine Fever virus (ASFV) infected animals, and more particularly pigs. Furthermore, present inventors have tested several dosage regimes and have found a particular dose range as well as a particular dosage regime for cyclic cidofovir or a prodrug thereof allowing treatment (including prevention of disease) of ASFV infected animals, and more particularly pigs. More particularly, present inventors found a dosage regime comprising two subsequent periods wherein the dose and/or timing of administration in the first period is different from the dose and/or timing in the second period, preferably wherein the average daily dose administered in the first period is higher than the average daily dose administered in the second period, reduced viral shedding and clinical signs of disease and improved survival in pigs challenged with ASFV, while allowing an acceptable or absent toxicity of cyclic cidofovir or a prodrug.

Accordingly, a first aspect provides cyclic cidofovir or a prodrug thereof for use in the treatment of African Swine Fever (ASF) in an animal comprising administering orally in a dose of from 10.0 to. 30.0 mg/kg, preferably from 15.0 to 25.0 mg/kg, such as about 20.0 mg/kg, of said cyclic cidofovir or prodrug thereof, wherein at least one dose of said cyclic cidofovir or prodrug thereof, such as one or two doses, is administered to the animal daily for a first period of from 1 to 4 consecutive days and one dose of cyclic cidofovir is administered to the animal every other day or daily for a second period of at least 5 consecutive days, such as from 5 to 14 consecutive days, wherein the average daily dose administered in the first period is higher than the average daily dose administered in the second period. Here, the “mg/kg” refers to the amount of cyclic cidofovir or prodrug thereof per kg body weight of the animal. Furthermore, the average daily dosage takes into account the total doses administered in that period and the total number of days in that period. For example, for a period of 5 consecutive days wherein 20.0 mg/kg of cyclic cidofovir is administered to the animal once every other day and wherein the period starts with a day of administration, the average daily dose is 12.0 mg/kg (i.e. (20.0 mg/kg*3)/5).

A further aspect provides a method of treating (defined herein as to encompass treating and preventing) ASF in an animal comprising administering orally in a dose of from 10.0 to. 30.0 mg/kg of cyclic cidofovir or prodrug thereof, wherein at least one dose of cyclic cidofovir, such as one or two doses, is administered to the animal daily for a first period of from 1 to 4 consecutive days and one dose of cyclic cidofovir is administered to the animal every other day or daily for a second period of at least 5 consecutive days, such as from 5 to 14 consecutive days, and wherein the average daily dose administered in the first period is higher than the average daily dose administered in the second period.

A further aspect provides the use of cyclic cidofovir or prodrug thereof in the preparation of a medicament for the treatment (defined herein as to encompass treatment and prevention) of ASF in an animal, wherein said cyclic cidofovir or prodrug thereof is to be administered orally in a dose of from 10.0 to. 30.0 mg/kg, wherein at least one dose of cyclic cidofovir, such as one or two doses, is administered to the animal daily for a first period of from 1 to 4 consecutive days and one dose of cyclic cidofovir is administered to the animal every other day or daily for a second period of at least 5 consecutive days, such as from 5 to 14 consecutive days, and wherein the average daily dose administered in the first period is higher than the average daily dose administered in the second period.

The term “cyclic cidofovir”, “cCDV”, “cyclic(S)-HPMPC”, or “cHPMPC” as used herein refers to a compound with the structural formula:

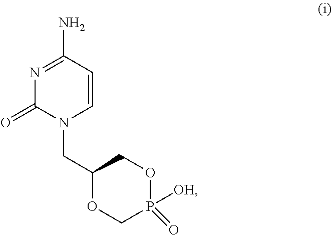

or any derivative thereof. cHPMPC is annotated under the PubChem database with PubChem CID 122873.

The term “cyclic cidofovir”, “cCDV”, or “cHPMPC” as used herein thus encompasses isomers, solvates, tautomers, salts, esters, amidates, esteramidates or pharmaceutically acceptable salts of the compound of the formula (i) hereabove or to compounds in equilibrium with the compound of the formula hereabove. The phosphonate function of a nucleoside analogue can be derivatized in order to increase the activity, the pharmacokinetic or -dynamic profile. Therefore, also encompassed by the term “cyclic cidofovir”, “cCDV”, or “cHPMPC” are the phosphonate esters, amidates or esteramidates of the compound of formula (i), wherein the phosphonate can be mono- or disubstituted. Examples of such esters comprise alkyl esters, alkenyl esters, alkynyl esters, alkoxyalkyl esters, alkoxyalkenyl esters such as octyl, tetracosyl, hexadecyloxypropyl, octadecyloxyethyl, oleyloxypropyl, tetradecyloxypropyl, octadecyloxypropyl, oleyloxyethyl, 1-O-octadecyl-2-O-benzyl-glyceryl and the like. A cyclic cidofovir can be prepared by any method known in the art, such as described for example in International patent application WO 9507919 A1.

Further suitable examples of esters, amidates or esteramidates of the compound of formula (i), include Acyloxyalkyl esters, Alkoxycarbonyloxyalkyl esters, Alkoxyalkyl esters, or amidates such as phosphoramidates or phosphonamidates, also known as ProTides.

The term “prodrug” as used herein refers to a medicament or compound that, after administration to a subject, is metabolized into a pharmacologically active compound. A prodrug may improve how the pharmacologically active compound is absorbed, distributed, metabolized and excreted (ADME) by the subject's body. Cyclic cidofovir is itself considered a prodrug of HPMPC (cidofovir).

Nevertheless further prodrugs could be considered. For instance, the prodrug of cyclic cidofovir may be a prodrug of cyclic cidofovir as described in Pertusat F. et al., Medicinal Chemistry of Nucleoside Phosphonate Prodrugs for Antiviral Therapy, Antiviral Chemistry and Chemotherapy, 2012, such as phenyl or salicylate ester prodrugs or a serine peptide phosphoester or alkyloxycarbonylphenyl prodrug of cyclic cidofovir or a peptide conjugate of cyclic cidofovir, such as described in International patent application WO 2006014429A2.

In particular embodiments, the cyclic cidofovir is selected from the group consisting of pivaloyloxymethyl cyclic cidofovir, isopropyloxycarbonyloxymethyl cyclic cidofovir, isopropyl cyclic cidofovir L-alaninate, isopropyl cyclic cidofovir L-valinate, 1-O-hexadecylpropanediol-3-cyclic cidofovir, 1-O-octadecylpropanediol-3-cyclic cidofovir, 1-O-octadecylethanediol-2-cyclic cidofovir, hexadecyloxypropyl cyclic cidofovir, octadecyloxyethyl cyclic cidofovir, oleyloxypropyl cyclic cidofovir, octyloxypropyl cyclic cidofovir, dodecyloxypropyl cyclic cidofovir, oleyloxyethyl cyclic cidofovir, 1-O-octadecyl-2-O-benzyl-glyceryl cyclic cidofovir, tetradecyloxypropyl cyclic cidofovir, eicosyl cyclic cidofovir, docosyl cyclic cidofovir, and hexadecyl cyclic cidofovir, preferably the cyclic cidofovir prodrug is selected from the group consisting of pivaloyloxymethyl cyclic cidofovir, isopropyloxycarbonyloxymethyl cyclic cidofovir, isopropyl cyclic cidofovir L-alaninate, and isopropyl cyclic cidofovir L-valinate.

In some embodiments the cyclic cidofovir for use according to the present invention has a structure of formula (I) or (II),

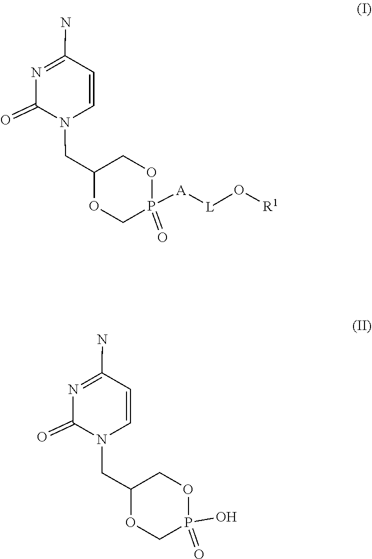

wherein,
A is selected from the group consisting of O and NH;
L is selected from the group consisting of —(CR²R³)ₙ—, —CH(R⁴)—C(O)— and —C(O)—CH(R⁵)—;
n is an integer selected from 1, 2, 3, 4 or 5;
R¹ is selected from the group consisting of C₆₋₂₅alkyl, —C(O)R⁶, —CO₂R⁷, C₆₋₂₅haloalkyl, C₆₋₂₅alkenyl, C₆₋₂₅haloalkenyl, C₃₋₁₂cycloalkyl and C₃₋₁₂halocycloalkyl;
each R² is independently selected from the group consisting of hydrogen, C₁₋₆alkyl, C₃₋₁₂cycloalkyl and halogen;
each R³ is independently selected from the group consisting of hydrogen, C₁₋₆alkyl, C₃₋₁₂cycloalkyl and halogen;
each R⁴ is independently selected from the group consisting of hydrogen, C₁₋₆alkyl, C₃₋₁₂cycloalkyl and halogen;
each R⁶ is independently selected from the group consisting of hydrogen, C₁₋₆alkyl, C₃₋₁₂cycloalkyl and halogen;
each R⁶ is independently selected from the group consisting of C₁₋₁₀alkyl, C₁₋₁₀haloalkyl, C₃₋₁₂cycloalkyl, C₃₋₁₂halocycloalkyl, C₂₋₁₀alkeny, and C₂₋₁₀haloalkenyl;
each R⁷ is independently selected from the group consisting of C₁₋₁₀alkyl, C₁₋₁₂haloalkyl, C₃₋₁₂cycloalkyl, C₃₋₁₂halocycloalkyl, C₂₋₁₀alkeny, and C₂₋₁₀haloalkenyl;
or a stereoisomer, tautomer, solvate, hydrate, pharmaceutically acceptable salt thereof.

The term “halo” or “halogen” as a group or part of a group is generic for fluoro, chloro, bromo, iodo.

The term “alkyl”, as a group or part of a group, refers to a hydrocarbyl group of formula —CₙH₂ₙ₊₁ wherein n is a number greater than or equal to 1. Alkyl groups may be linear or branched and may be substituted as indicated herein. Generally, alkyl groups of this invention comprise up to 25 carbon atoms, preferably up to 24 carbon atoms, preferably up to 23 carbon atoms, more preferably up to 22 carbon atoms, still more preferably up to 21 carbon atoms. When a subscript is used herein following a carbon atom, the subscript refers to the number of carbon atoms that the named group may contain.

For example, “C₁₋₆alkyl” includes all linear or branched alkyl groups with between 1 and 6 carbon atoms, and thus includes methyl, ethyl, n-propyl, i-propyl, butyl and its isomers (e.g. n-butyl, i-butyl and t-butyl); pentyl and its isomers, hexyl and its isomers. For example, “C₁₋₅alkyl” includes all includes all linear or branched alkyl groups with between 1 and 5 carbon atoms, and thus includes methyl, ethyl, n-propyl, i-propyl, butyl and its isomers (e.g. n-butyl, i-butyl and t-butyl); pentyl and its isomers.

For example, “C₁₋₄alkyl” includes all linear or branched alkyl groups with between 1 and 4 carbon atoms, and thus includes methyl, ethyl, n-propyl, i-propyl, butyl and its isomers (e.g. n-butyl, i-butyl and t-butyl). For example “C₁₋₃alkyl” includes all linear or branched alkyl groups with between 1 and 3 carbon atoms, and thus includes methyl, ethyl, n-propyl, i-propyl.

When the term “alkyl” is used as a suffix following another term, as in “hydroxyalkyl,” this is intended to refer to an alkyl group, as defined above, being substituted with one or two (preferably one) substituent(s) selected from the other, specifically-named group, also as defined herein. The term “hydroxyalkyl” therefore refers to a —R^{a}—OH group wherein R^{a} is alkylene as defined herein.

The term “haloalkyl” as a group or part of a group, refers to an alkyl group having the meaning as defined above wherein one or more hydrogen atoms are each replaced with a halogen as defined herein. Non-limiting examples of such haloalkyl groups include chloromethyl, 1-bromoethyl, fluoromethyl, difluoromethyl, trifluoromethyl, 1,1,1-trifluoroethyl, trichloromethyl, tribromomethyl, and the like.

The term “cycloalkyl”, as a group or part of a group, refers to a cyclic alkyl group, that is a monovalent, saturated, hydrocarbyl group having 1 or more cyclic structure, and comprising from 3 to 12 carbon atoms, more preferably from 3 to 9 carbon atoms, more preferably from 3 to 7 carbon atoms; more preferably from 3 to 6 carbon atoms. Cycloalkyl includes all saturated hydrocarbon groups containing 1 or more rings, including monocyclic or bicyclic groups. The further rings of multi-ring cycloalkyls may be either fused, bridged and/or joined through one or more spiro atoms. When a subscript is used herein following a carbon atom, the subscript refers to the number of carbon atoms that the named group may contain. For example, the term “C₃₋₈cycloalkyl”, a cyclic alkyl group comprising from 3 to 8 carbon atoms. For example, the term “C₃₋₆cycloalkyl”, a cyclic alkyl group comprising from 3 to 6 carbon atoms. Examples of C₃₋₁₂cycloalkyl groups include but are not limited to cyclopropyl, cyclobutyl, cyclopentyl, cyclohexyl, cycloheptyl, cyclooctyl, bicycle[2.2.1]heptan-2yl, (1S,4R)-norbornan-2-yl, (1R,4R)-norbornan-2-yl, (1S,4S)-norbornan-2-yl, (1R,4S)-norbornan-2-yl, 1-adamantyl.

The term “halocycloalkyl”, as a group or part of a group, refers to a cyclalkyl group having the meaning as defined above wherein one, or more hydrogen atoms are each replaced with a halogen as defined herein.

The term “alkenyl” as a group or part of a group, refers to an unsaturated hydrocarbyl group, which may be linear, or branched, comprising one or more carbon-carbon double bonds. When a subscript is used herein following a carbon atom, the subscript refers to the number of carbon atoms that the named group may contain. For example, the term “C₂₋₆alkenyl” refers to an unsaturated hydrocarbyl group, which may be linear, or branched comprising one or more carbon-carbon double bonds and comprising from 2 to 6 carbon atoms. For example, C₂₄alkenyl includes all linear, or branched alkenyl groups having 2 to 4 carbon atoms. Examples of C₂₋₆alkenyl groups are ethenyl, 2-propenyl, 2-butenyl, 3-butenyl, 2-pentenyl and its isomers, 2-hexenyl and its isomers, 2,4-pentadienyl. and the like.

The term “haloalkenyl” as a group or part of a group, refers to a alkenyl group having the meaning as defined above wherein one or more hydrogen atoms are each replaced with a halogen as defined herein.

The compounds of the invention may also exist in unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

A currently accepted classification system for organic hydrates is one that defines isolated site, channel, or metal-ion coordinated hydrates—see Polymorphism in Pharmaceutical Solids by K. R.

Morris (Ed. H. G. Britain, Marcel Dekker, 1995), incorporated herein by reference. Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

As used herein and unless otherwise stated, the term “stereoisomer” refers to all possible different isomeric as well as conformational forms which the compounds of structural formula herein may possess, in particular all possible stereochemically and conformationally isomeric forms, all diastereomers, enantiomers and/or conformers of the basic molecular structure. Some compounds of the present invention may exist in different tautomeric forms, all of the latter being included within the scope of the present invention.

The present invention includes all possible stereoisomers compounds of formula (I) and any subgroup thereof. When a compound is desired as a single enantiomer, such may be obtained by stereospecific synthesis, by resolution of the final product or any convenient intermediate, or by chiral chromatographic methods as each are known in the art. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994), incorporated by reference with regard to stereochemistry. A structural isomer is a type of isomer in which molecules with the same molecular formula have different bonding patterns and atomic organization. Where structural isomers are interconvertible via a low energy barrier, tautomeric isomerism (‘tautomerism’) can occur. This can take the form of proton tautomerism in compounds of the invention containing, for example, an imino, keto, or oxime group, or so-called valence tautomerism in compounds which contain an aromatic moiety.

The compounds of the invention may be in the form of salts, preferably pharmaceutically acceptable salts, as generally described below. Some preferred, but non-limiting examples of suitable pharmaceutically acceptable organic and/or inorganic acids are as hydrochloric acid, hydrobromic acid, sulfuric acid, nitric acid, acetic acid and citric acid, as well as other pharmaceutically acceptable acids known per se (for which reference is made to the prior art referred to below).

When the compounds of the invention contain an acidic group as well as a basic group the compounds of the invention may also form internal salts, and such compounds are within the scope of the invention. When the compounds of the invention contain a hydrogen-donating heteroatom (e.g. NH), the invention also covers salts and/or isomers formed by transfer of said hydrogen atom to a basic group or atom within the molecule.

Pharmaceutically acceptable salts of the compounds of formula (1) and any subgroup thereof include the acid addition and base salts thereof. Suitable acid addition salts are formed from acids which form non-toxic salts. Examples include the acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002), incorporated herein by reference.

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

Pharmaceutically acceptable salts of compounds of formula (1) may be prepared by one or more of these methods:
(i) by reacting the compound of formula (1) with the desired acid;
(ii) by reacting the compound of formula (1) with the desired base;
(iii) by removing an acid- or base-labile protecting group from a suitable precursor of the compound of formula (1) or by ring-opening a suitable cyclic precursor, for example, a lactone or lactam, using the desired acid; or
(iv) by converting one salt of the compound of formula (1) to another by reaction with an appropriate acid or by means of a suitable ion exchange column.

All these reactions are typically carried out in solution. The salt may precipitate from solution and be collected by filtration or may be recovered by evaporation of the solvent. The degree of ionization in the salt may vary from completely ionized to almost non-ionized.

In addition, although generally, with respect to the salts of the compounds of the invention, pharmaceutically acceptable salts are preferred, it should be noted that the invention in its broadest sense also included non-pharmaceutically acceptable salts, which may for example be used in the isolation and/or purification of the compounds of the invention.

In some embodiments the cyclic cidofovir for use according to the present invention has a structure of formula (1) as defined herein,
wherein,
A is selected from the group consisting of O and NH;
L is selected from the group consisting of —(CR²R³)ₙ—, —CH(R⁴)—C(O)— and —C(O)—CH(R⁵)—; preferably L is —(CR²R³)ₙ— or —CH(R⁴)—C(O)—;
n is an integer selected from 1, 2, 3, 4 or 5; preferably n is 1, 2, 3, or 4; preferably n is 1, 2, or 3;
R¹ is selected from the group consisting of C₆₋₂₅alkyl, —C(O)R⁶, —CO₂R⁷, C₆₋₂₅haloalkyl, C₆₋₂₅alkenyl, C₆₋₂₅ haloalkenyl, C₃₋₁₂cycloalkyl and C₃₋₁₂halocycloalkyl; preferably R¹ is C₆₋₂₄alkyl, —C(O)R⁶, —CO₂R⁷, C₆₋₂₄ haloalkyl, C₆₋₂₅alkenyl, C₆₋₂₄haloalkenyl, C₃₋₁₂cycloalkyl and C₃₋₁₂halocycloalkyl; preferably R¹ is C₆₋₂₄ alkyl, —C(O)R⁶, —CO₂R⁷, C₆₋₂₄haloalkyl, C₆₋₂₄alkenyl, C₆₋₂₄haloalkenyl, C₃₋₁₀cycloalkyl and C₃₋₁₀halocycloalkyl; preferably R¹ is C₆₋₂₃alkyl, —C(O)R⁶, —CO₂R⁷, C₆₋₂₃haloalkyl, C₆₋₂₃alkenyl, C₆₋₂₃haloalkenyl, C₃₋₁₀cycloalkyl and C₃₋₁₀halocycloalkyl; preferably R¹ is C₆₋₂₂alkyl, —C(O)R⁶, —CO₂R⁷, C₆₋₂₂haloalkyl, C₆₋₂₂ alkenyl, C₆₋₂₂haloalkenyl, C₃₋₁₂cycloalkyl and C₃₋₁₂halocycloalkyl; preferably R¹ is C₆₋₂₁alkyl, —C(O)R⁶, —CO₂R⁷, C₆₋₂₁haloalkyl, C₆₋₂₁alkenyl, C₆₋₂₁haloalkenyl, C₃₋₁₀cycloalkyl and C₃₋₁₀halocycloalkyl; preferably R¹ is C₆₋₂₀alkyl, —C(O)R⁶, —CO₂R⁷, C₆₋₂₀haloalkyl, C₆₋₂₀alkenyl, C₆₋₂₀haloalkenyl, C₃₋₁₂cycloalkyl and C₃₋₁₂halocycloalkyl; preferably R¹ is C₆₋₂₀alkyl, —C(O)R⁶, —CO₂R⁷, C₆₋₂₀haloalkyl, C₆₋₂₀alkenyl, C₆₋₂₀haloalkenyl, C₃₋₁₀cycloalkyl and C₃₋₁₀halocycloalkyl; preferably R¹ is C₆₋₂₀alkyl, —C(O)R⁶, —CO₂R⁷, C₆₋₂₀haloalkyl, C₃₋₁₂cycloalkyl and C₃₋₁₂halocycloalkyl; preferably preferably R¹ is C₆₋₂₀alkyl, —C(O)R⁶, and —CO₂R⁷; preferably R¹ is C₆₋₁₈alkyl, —C(O)R⁶, and —CO₂R⁷;
each R² is independently selected from the group consisting of hydrogen, C₁₋₆alkyl, C₃₋₁₂cycloalkyl and halogen; preferably each R² is independently hydrogen, C₁₋₆alkyl, C₃₋₁₀cycloalkyl and halogen; preferably each R² is independently hydrogen, C₁₋₆alkyl, C₃₋₁₀cycloalkyl, fluoro, chloro, iodo, and bromo; preferably each R² is independently hydrogen, C₁₋₅alkyl, C₃₋₁₀cycloalkyl, fluoro, chloro, iodo, and bromo; preferably each R² is independently hydrogen, C₁₋₅alkyl, C₃₋₈cycloalkyl, fluoro, chloro, iodo, and bromo; preferably each R² is independently hydrogen, and C₁₋₅alkyl;
each R³ is independently selected from the group consisting of hydrogen, C₁₋₆alkyl, C₃₋₁₂cycloalkyl and halogen; preferably each R³ is independently hydrogen, C₁₋₆alkyl, C₃₋₁₀cycloalkyl and halogen; preferably each R³ is independently hydrogen, C₁₋₆alkyl, C₃₋₁₀cycloalkyl, fluoro, chloro, iodo, and bromo; preferably each R³ is independently hydrogen, C₁₋₅alkyl, C₃₋₁₀cycloalkyl, fluoro, chloro, iodo, and bromo; preferably each R³ is independently hydrogen, C₁₋₅alkyl, C₃₋₈cycloalkyl, fluoro, chloro, iodo, and bromo; preferably each R³ is independently hydrogen, and C₁₋₅alkyl;
each R⁴ is independently selected from the group consisting of hydrogen, C₁₋₆alkyl, C₃₋₁₂cycloalkyl and halogen; preferably each R⁴ is independently hydrogen, C₁₋₆alkyl, C₃₋₁₀cycloalkyl and halogen; preferably each R⁴ is independently hydrogen, C₁₋₆alkyl, C₃₋₁₀cycloalkyl, fluoro, chloro, iodo, and bromo; preferably each R⁴ is independently hydrogen, C₁₋₅alkyl, C₃₋₁₀cycloalkyl, fluoro, chloro, iodo, and bromo; preferably each R⁴ is independently hydrogen, C₁₋₅alkyl, C₃₋₈cycloalkyl, fluoro, chloro, iodo, and bromo; preferably each R⁴ is independently hydrogen, C₁₋₄alkyl, and C₃₋₈cycloalkyl; preferably each R⁴ is independently hydrogen, and C₁₋₅alkyl;
each R⁶ is independently selected from the group consisting of hydrogen, C₁₋₆alkyl, C₃₋₁₂cycloalkyl and halogen; preferably each R⁶ is independently hydrogen, C₁₋₆alkyl, C₃₋₁₀cycloalkyl and halogen; preferably each R⁶ is independently hydrogen, C₁₋₆alkyl, C₃₋₁₀cycloalkyl, fluoro, chloro, iodo, and bromo; preferably each R⁶ is independently hydrogen, C₁₋₅alkyl, C₃₋₁₀cycloalkyl, fluoro, chloro, iodo, and bromo; preferably each R⁵ is independently hydrogen, C₁₋₅alkyl, C₃₋₈cycloalkyl, fluoro, chloro, iodo, and bromo; preferably each R⁵ is independently hydrogen, and C₁₋₅alkyl;
each R⁶ is independently selected from the group consisting of C₁₋₁₀alkyl, C₁₋₁₀haloalkyl, C₃₋₁₂cycloalkyl, C₃₋₁₂halocycloalkyl, C₂₋₁₀alkeny, and C₂₋₁₀haloalkenyl; preferably each R⁶ is independently C₁₋₅alkyl, C₁₋₈ haloalkyl, C₃₋₁₀cycloalkyl, C₃₋₁₀halocycloalkyl, C₂₋₈alkeny, and C₂₋₈haloalkenyl; preferably each R⁶ is independently C₁₋₆alkyl, C₁₋₆haloalkyl, C₃₋₈cycloalkyl, C₃₋₈halocycloalkyl, C₂₋₆alkeny, and C₂₋₈haloalkenyl; preferably each R⁶ is independently C₁₋₆alkyl, C₁₋₆haloalkyl, C₂₋₆alkeny, and C₂₋₆haloalkenyl; each R⁷ is independently selected from the group consisting of C₁₋₁₀alkyl, C₁₋₁₂haloalkyl, C₃₋₁₂cycloalkyl, C₃₋₁₂halocycloalkyl, C₂₋₁₀alkeny, and C₂₋₁₀haloalkenyl; preferably each R⁷ is independently C₁₋₅alkyl, C₁₋₈ haloalkyl, C₃₋₁₀cycloalkyl, C₃₋₁₀halocycloalkyl, C₂₋₈alkeny, and C₂₋₈haloalkenyl; preferably each R⁷ is independently C₁₋₆alkyl, C₁₋₆haloalkyl, C₃₋₈cycloalkyl, C₃₋₈halocycloalkyl, C₂₋₆alkeny, and C₂₋₈haloalkenyl; preferably each R⁷ is independently C₁₋₆alkyl, C₁₋₆haloalkyl, C₂₋₆alkeny, and C₂₋₆haloalkenyl; or a stereoisomer, tautomer, solvate, hydrate, pharmaceutically acceptable salt thereof.

In some embodiments A is O. In some embodiments L is —(CR²R³)ₙ—. In some embodiments L is —CH(R⁴)—.

Particularly preferred compounds of the invention are those compounds listed in the Table below.

| ID | Structure |
|---|---|
| cHPMPC | 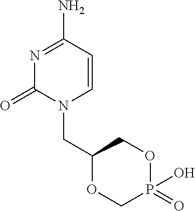 |
| 1 | 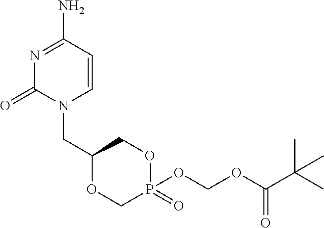 |
| 2 | 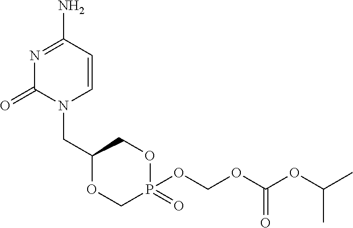 |
| 3 | 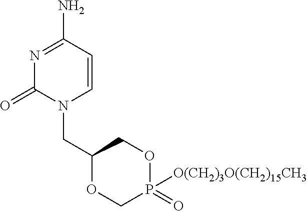 |
| 4 | 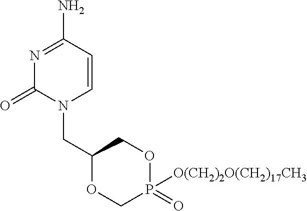 |
| 5 | 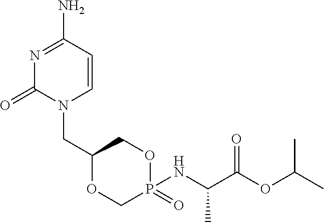 |

The terms “treat” or “treatment” encompass both the therapeutic treatment of an already developed disease or condition, such as the therapy of an already developed ASFV infection, as well as prophylactic or preventive measures, wherein the aim is to prevent or lessen the chances of incidence of an undesired affliction, such as to prevent occurrence, development and progression of an ASFV infection. Beneficial or desired clinical results may include, without limitation, alleviation of one or more symptoms or one or more biological markers, diminishment of extent of disease, stabilised (i.e., not worsening) state of disease, delay or slowing of disease progression, amelioration or palliation of the disease state, and the like. “Treatment” can also mean prolonging survival as compared to expected survival if not receiving treatment. “Treatment” can also mean decreasing the viral load of ASFV in an animal, thereby reducing infectivity and/or decreasing the viral shedding in an animal, thereby reducing the transmission/spread of the virus/disease.

Except when noted, the terms “animal”, “subject” or “patient” can be used interchangeably and refer to animals, preferably warm-blooded animals, more preferably vertebrates, even more preferably mammals, still more preferably a member of the family Suidae, still more preferably a member of the genus Sus. Preferred subjects are wild or domestic pigs or swines. More preferred subjects are domestic pigs (Sus domesticus). Non-limiting examples of members of the family Suidae include Golden babirusa, Sulawesi babirusa, Togian babirusa, Giant forest hog, Desert warthog, common warthog, pygmy hog, bushpig, red river hog, Palawan bearded pig, bearded pig, Vietnamese warty pig, Visayan warty pig, Celebes warty pig, Flores warty pig, Mindoro warty pig, Philippine warty, Javan warty pig, wild boar and domestic pig.

The terms “subject” or “patient” include subjects in need of treatment, more particularly subjects that would benefit from treatment of a given condition, particularly a ASFV infection. Such subjects may include, without limitation, those that have been diagnosed with said condition, those prone to develop said condition and/or those in who said condition is to be prevented.

In particular embodiments, the animal is a member of the family Suidae, a member of the genus Sus, preferably a domestic pig, wild pig, domestic swine or wild swine (i.e. wild boar).

The term “African swine fever” or “ASF” as used herein refers to a viral disease caused by an infection with an ASFV. The ASFV is a large, enveloped, double-stranded DNA virus. Its genome comprises 170-193 kilobase pairs with up to 167 open reading frames. ASFV is the sole member of the family Asfarviridae, and the only known DNA transmitted by arthropods, namely soft ticks of the genus Ornithodoros. Based on sequence variation in the C-terminal region of the B646L gene encoding the major capsid protein p72, 24 ASFV genotypes (I-XXIV) have been identified so far. ASF may be any type of ASF, including ASF caused by any ASFV genotype or ASFV isolate, such as the Georgia 2007/1 isolate, the KAB6/2 isolate, the BOT1/99 isolate and the Magadi w/hog 9 isolate.

In particular embodiments, the cyclic cidofovir or prodrug thereof allows to treat pan-genotype ASFV.

In particular embodiments, the cyclic cidofovir or prodrug thereof is administered orally in the form of a powder, a liquid, a semi-liquid, a pill, a tablet, a capsule, or a granule.

Daily administration of a dose of cyclic cidofovir or prodrug thereof to an animal may refer to administration of the entire dose of cyclic cidofovir or prodrug thereof at one given moment during the day (i.e. once daily). Daily administration of a dose of cyclic cidofovir or prodrug thereof to an animal also encompasses the administration of the dose of cyclic cidofovir or prodrug thereof spread over multiple given moments during the day or over a prolonged period of time during the day, such as by continuous administration, for instance by allowing ad libitum access to a source of water and/or feed containing an adjusted dosage.

If two doses of cyclic cidofovir or prodrug thereof are to be administered to the animal daily (wherein the daily dose of cyclic cidofovir or prodrug thereof is double the daily dose as for the administration of one dose of cyclic cidofovir or prodrug thereof), each of these two doses may be administered to the animal at one given moment during the day (e.g. twice a day as described elsewhere herein), or the administration of each dose may be spread over multiple given moments during the day or over a prolonged period of time during the day, such as by continuous or ad libitum administration.

In particular embodiments, the cyclic cidofovir or prodrug thereof is administered orally by admixing the cyclic cidofovir or prodrug thereof with the feed or drinking water of the animal.

In particular embodiments, the cyclic cidofovir or prodrug thereof is administered to the animal in a dose of from 10.0 to. 30.0 mg of cyclic cidofovir or prodrug thereof per kg bodyweight of the animal (mg/kg), from 15.0 to 30.0 mg/kg, from 20.0 to 30.0 mg/kg, or from 25.0 to 30.0 mg/kg, preferably about 30.0 mg/kg, of said cyclic cidofovir or prodrug thereof.

On a daily basis, one or two of such doses of cyclic cidofovir or prodrug thereof may be administered to the animal. In particular embodiments, if one dose of cyclic cidofovir or prodrug thereof is administered to the animal daily, the total daily dose or total amount of cyclic cidofovir or prodrug thereof is from 10.0 to. 30.0 mg of cyclic cidofovir or prodrug thereof per kg bodyweight of the animal (mg/kg), from 15.0 to 30.0 mg/kg, from 20.0 to 30.0 mg/kg, or from 25.0 to 30.0 mg/kg, preferably about 30.0 mg/kg, of said cyclic cidofovir or prodrug thereof. In particular embodiments, if two doses of cyclic cidofovir or prodrug thereof are administered to the animal daily, the total daily dose of cyclic cidofovir or prodrug thereof is from 20.0 to. 60.0 mg of cyclic cidofovir or prodrug thereof per kg bodyweight of the animal (mg/kg), from 30.0 to 60.0 mg/kg, from 40.0 to 60.0 mg/kg, or from 50.0 to 60.0 mg/kg, preferably about 60.0 mg/kg, of said cyclic cidofovir or prodrug thereof. Similarly, if three or more doses are administered, this will correspond to a total dosage of three or more times from 10.0 to. 30.0 mg of cyclic cidofovir or prodrug thereof per kg bodyweight of the animal (mg/kg), from 15.0 to 30.0 mg/kg, from 20.0 to 30.0 mg/kg, or from 25.0 to 30.0 mg/kg, preferably about 30.0 mg/kg, of said cyclic cidofovir or prodrug thereof.

In particular embodiments, at least one dose of cyclic cidofovir or the prodrug thereof, such as one or two doses, is administered to the animal daily for (i.e. during) a first period of from 1 to 4 consecutive days and one dose of cyclic cidofovir is administered to the animal every other day or daily for a second period of from 5 to 14 consecutive days, preferably from 5 to 7 consecutive days, such as 5 consecutive days. Accordingly, in particular embodiments, a total daily dose of from 10.0 to. 60.0 mg/kg of cyclic cidofovir or the prodrug thereof is administered to the animal daily for a first period of from 1 to 4 consecutive days and a total daily dose of from 10.0 to. 30.0 mg/kg of cyclic cidofovir or the prodrug thereof is administered to the animal every other day or daily for a second period of from 5 to 14 consecutive days, preferably from 5 to 7 consecutive days, such as 5 consecutive days.

In particular embodiment, the second administration period is immediate subsequent to the first administration period, meaning that the first and second administration periods are not separated by a third period of administration or no administration of the cyclic cidofovir or prodrug thereof.

In particular embodiments, the administration comprises a first period of from 1 to 4 consecutive days, such as 1, 2, 3 or 4 consecutive days, preferably 3 consecutive days wherein two doses of cyclic cidofovir or prodrug thereof are administered to the animal daily.

In particular embodiments, the administration comprises a first period of from 1 to 4 consecutive days, such as 1, 2, 3 or 4 consecutive days, preferably 3 consecutive days wherein one dose of cyclic cidofovir or prodrug thereof is administered to the animal daily.

In particular embodiments, the administration comprises a second period of from 5 to 14 consecutive days, such as from 5 to 13 consecutive days, from 5 to 12 consecutive days, from 5 to 10 consecutive days, from 5 to 8 consecutive days, from 5 to 7 consecutive days, preferably 5 days, wherein one dose of the cyclic cidofovir or prodrug thereof is administered to the animal daily.

In particular embodiments, the administration comprises a second period of from 5 to 14 consecutive days, preferably from 5 to 7 consecutive days, such as 5, 6 or 7 consecutive days, more preferably 5 consecutive days, or preferably from 10 to 14 consecutive days, such as 10, 11, 12, 13 or 14 consecutive days, more preferably 13 consecutive days, wherein one dose of the cyclic cidofovir or prodrug thereof is administered to the animal every other day.

In particular embodiments, one dose of cyclic cidofovir is administered to the animal every other day or daily for a second period of from 5 to 7 consecutive days, preferably 5 consecutive days.

In particular embodiment, the second period wherein the cyclic cidofovir or prodrug thereof is administered every other day, starts with an administration (i.e. treatment) day.

In particular embodiments, said administration comprises a first period of 3 consecutive days wherein two doses of the cyclic cidofovir or prodrug thereof are administered to the animal daily and a second period of from 5 to 14 consecutive days, such as from 5 to 12 consecutive days, from 5 to 10 consecutive days, from 5 to 8 consecutive days, from 5 to 7 consecutive days, preferably 5 consecutive days, wherein one dose of the cyclic cidofovir or prodrug thereof is administered to the animal daily.

In particular embodiments, one dose of said cyclic cidofovir or prodrug thereof is administered to the animal daily for a first period of 3 consecutive days and one dose of said cyclic cidofovir or prodrug thereof is administered to the animal every other day for a second period of from 5 to 14 consecutive days, preferably from 5 to 7 consecutive days, such as 5, 6 or 7 consecutive days, more preferably 5 consecutive days, or preferably from 10 to 14 consecutive days, such as 10, 11, 12, 13 or 14 consecutive days, more preferably 13 consecutive days.

The present inventors have found that the dosage regime comprising a first period wherein two doses of the cyclic cidofovir or prodrug thereof are administered to the animal daily and a second period wherein one dose of the cyclic cidofovir or prodrug thereof is administered to the animal daily, as described elsewhere herein, is preferred over a dosage regime comprising the administration of one dose of cyclic cidofovir or prodrug thereof to the animal daily for a first period and the administration of one dose of said cyclic cidofovir or prodrug thereof to the animal every other day for a second period, as described elsewhere herein.

As described elsewhere herein, the dose of cyclic cidofovir or prodrug thereof may be administered to the animal entirely at one given moment in time.

Accordingly, in particular embodiments, the administration comprises a first period of from 1 to 4 consecutive days, such as 1, 2, 3 or 4 consecutive days, preferably 3 consecutive days, wherein two doses of cyclic cidofovir or prodrug thereof are administered to the animal daily and wherein each of said doses of cyclic cidofovir or prodrug thereof is administered entirely to the animal at one given moment in time. In such embodiment, the animal will receive twice a day one administration of one dose of cyclic cidofovir or prodrug thereof, resulting in a total of two doses per day.

If the animal receives two doses per day, each dose of from 10.0 to. 30.0 mg/kg cyclic cidofovir or prodrug thereof is preferably the same.

In particular embodiments, the administration comprises a first period of from 1 to 4 consecutive days, such as 1, 2, 3 or 4 consecutive days, preferably 3 consecutive days wherein a dose of cyclic cidofovir or prodrug thereof is administered to the animal once a day. Accordingly, in such embodiment, the animal receives at one given moment during the day the entire dose of cyclic cidofovir or prodrug thereof.

In particular embodiments, the administration comprises a second period of from 5 to 14 consecutive days, such as from 5 to 12 consecutive days, from 5 to 10 consecutive days, from 5 to 8 consecutive days, from 5 to 7 consecutive days, preferably five days, wherein one dose of cyclic cidofovir or prodrug thereof is administered to the animal once a day. Accordingly, in such embodiment, the animal receives at one given moment during the day the entire dose of cyclic cidofovir or prodrug thereof.

In particular embodiments, said administration comprises a second period of from 5 to 14 consecutive days, preferably from 5 to 7 consecutive days, such as 5, 6 or 7 consecutive days, more preferably 5 consecutive days, or preferably from 10 to 14 consecutive days, such as 10, 11, 12, 13 or 14 consecutive days, more preferably 13 consecutive days, wherein one dose of cyclic cidofovir or prodrug thereof is administered to the animal once every other day. Accordingly, in such embodiment, the animal receives at one given moment during the day the entire dose of cyclic cidofovir or prodrug thereof.

In particular embodiment, the second period wherein the cyclic cidofovir or prodrug thereof is administered once every other day, starts with a treatment day.

In particular embodiments, said administration comprises a first period of 3 consecutive days wherein each dose of two doses of cyclic cidofovir or prodrug thereof is administered to the animal once a day (i.e. the animal receives twice a day one dose of cyclic cidofovir or prodrug thereof) and a second period of from 5 to 14 consecutive days, such as from 5 to 12 consecutive days, from 5 to 10 consecutive days, from 5 to 8 consecutive days, from 5 to 7 consecutive days, preferably 5 consecutive days, wherein one dose of cyclic cidofovir or prodrug thereof is administered to the animal once a day.

In particular embodiments, one dose of cyclic cidofovir or prodrug thereof is administered to the animal once a day for a first period of 3 consecutive days and one dose of cyclic cidofovir or prodrug thereof is administered to the animal once every other day for a second period of from 5 to 14 consecutive days, preferably from 5 to 7 consecutive days, such as 5, 6 or 7 consecutive days, more preferably 5 consecutive days, or preferably from 10 to 14 consecutive days, such as 10, 11, 12, 13 or 14 consecutive days, more preferably 13 consecutive days.

In particular embodiments, if administration occurs every day, the dose of cyclic cidofovir or prodrug thereof is preferably administered with from 20 to 28 hours, preferably from 22 to 26 hours, more preferably 24 hours, between two administrations. In particular embodiments, if administration occurs every day, the dose of cyclic cidofovir or prodrug thereof is preferably administered every day at approximately the same hour.

In particular embodiments, administration of the cyclic cidofovir or prodrug thereof coincides with the feeding time(s) of the animal.

In particular embodiments, if administration occurs every other day, the dose cyclic cidofovir or prodrug thereof is preferably administered with from 44 to 52 hours, preferably from 46 to 50 hours, more preferably 48 hours, between two administrations. In particular embodiments, if administration occurs every other day, the dose of cyclic cidofovir or prodrug thereof is preferably administered every other day at approximately the same hour.

In particular embodiments, if the animal receives two doses of cyclic cidofovir or prodrug thereof per day, the cyclic cidofovir or prodrug thereof is preferably administered with from 8 to 16 hours, preferably from 10 to 14 hours, more preferably 12 hours, between the administration of each of the two doses.

In particular embodiments, cyclic cidofovir or a prodrug thereof is administered to the animal when the animal is infected with ASFV or when infection with ASFV is to be prevented in said animal.

In particular embodiments, cyclic cidofovir or a prodrug thereof is administered to the animal as soon as one animal is diagnosed with ASF, wherein said animal diagnosed with ASF is housed in the same farm as the animal to be treated or in an area of 3-km to 10-km radius surrounding the housing of animal to be treated.

Upon infection of an animal with ASF, different zones and areas may be designated surrounding the infected zone (i.e. the zone that immediately surrounds an infected premises or infected animal) to control and monitor the outbreak. Immediately surrounding the infected zone, there may be an ASF buffer zone. Immediately surrounding the buffer zone, there may be an ASF surveillance zone.

However, different competent authorities may use different terms to refer to said zones. For example, the infection zone may also be referred to as containment zone. Furthermore, the buffer and/or surveillance zone may be a protection zone, being a zone where specific biosecurity and sanitary measures are implemented to prevent the entry of ASFV into a free country or zone from a neighboring country or zone of a different health status.

In particular embodiments, the animal is housed in an ASF infection, buffer, surveillance, protection or containment zone. The requirements of ASF infection, buffer, surveillance, protection or containment zones may be regulated by national competent authorities or by the world organization for animal health (OIE). Preferably, the width of the zone closest to the infected premise or infected animal is from 3-km to 20-km radius around the infected premise or infected animal. In particular embodiments, the zone typically has a perimeter of at least 3 km around the perimeter of the closest infected premises or animal.

Present inventors found that cyclic cidofovir or a prodrug thereof can be used to reduce viremia in and viral shedding from exposed pigs and to prophylactically treat healthy pigs in outbreak infection zones. Accordingly, in particular embodiments, the administration of said cyclic cidofovir or prodrug thereof prevents the spread of ASF in an animal population, such as within subsequent ASF buffer, surveillance, and/or protection containment zones.

The compounds of the invention may be employed in combination with other therapeutic agents for the treatment or prophylaxis of the infections or conditions indicated above.

In particular embodiments, cyclic cidofovir or the prodrug thereof is comprised in a pharmaceutical composition. The pharmaceutical composition may comprise a pharmaceutically acceptable carrier.

Such pharmaceutical formulations or compositions may be comprised in a kit of parts.

The term “pharmaceutically acceptable” as used herein is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, antioxidants, tonicity controlling agents, absorption delaying agents, and the like. The use of such media and agents for pharmaceutical active substances is well known in the art. Except insofar as any conventional media or agent is incompatible with the active substance, its use in the therapeutic compositions may be contemplated.

For example, for oral administration, pharmaceutical compositions may be formulated in the form of powders, pills, tablets, lacquered tablets, coated (e.g., sugar-coated) tablets, granules, hard and soft gelatin capsules, aqueous, alcoholic or oily solutions, syrups, emulsions or suspensions. In an example, without limitation, preparation of oral dosage forms may be is suitably accomplished by uniformly and intimately blending together a suitable amount of cyclic cidofovir or prodrug thereof as disclosed herein in the form of a powder, optionally also including finely divided one or more solid carrier, and formulating the blend in a pill, tablet or a capsule. Exemplary but non-limiting solid carriers include calcium phosphate, magnesium stearate, talc, sugars (such as, e.g., glucose, mannose, lactose or sucrose), sugar alcohols (such as, e.g., mannitol), dextrin, starch, gelatin, cellulose, polyvinylpyrrolidine, low melting waxes and ion exchange resins. Compressed tablets containing the pharmaceutical composition can be prepared by uniformly and intimately mixing cyclic cidofovir or prodrug thereof as disclosed herein with a solid carrier such as described above to provide a mixture having the necessary compression properties, and then compacting the mixture in a suitable machine to the shape and size desired. Moulded tablets may be made by moulding in a suitable machine, a mixture of powdered compound moistened with an inert liquid diluent. Suitable carriers for soft gelatin capsules and suppositories are, for example, fats, waxes, semisolid and liquid polyols, natural or hardened oils, etc.

In particular embodiments, the cyclic cidofovir or prodrug thereof is comprised in a pharmaceutical composition, preferably wherein said pharmaceutical composition is an aqueous solution.

In particular embodiments, the pH of the pharmaceutical composition is from 6.0 to 8.0. The pH may be neutralized by any method known in the art, such as by the inclusion of NaHCO₃.

In particular embodiments, the pharmaceutical composition is an aqueous solution comprising from 5 to 40 mg/ml, preferably from 10 to 30 mg/ml, such as about 20 mg/ml, of cyclic cidofovir or prodrug thereof.

A further aspect provides a water solution comprising water and cyclic cidofovir or prodrug thereof.

The person skilled in the art will understand that the concentration of cyclic cidofovir or prodrug in the water solution can be adjusted depending on the type of animal, the age of the animal and/or the type of animal farm (e.g. breeding/nursery facility, weaner farm, fattening farm) as long as the final daily dose of cyclic cidofovir or prodrug consumed by the animal is as described elsewhere herein.

A further aspect provides a feed composition comprising feed and cyclic cidofovir or prodrug thereof. The person skilled in the art will understand that the concentration of cyclic cidofovir or prodrug in the feed can be adjusted depending on the type of animal, the age of the animal and/or the type of animal farm (e.g. breeding/nursery facility, weaner farm, fattening farm) as long as the final daily dose of cyclic cidofovir or prodrug consumed by the animal is as described elsewhere herein.

In particular embodiments, the pharmaceutical composition, water solution or feed composition as provided herein comprises an an ester, an amidate or esteramidate of a compound of formula (i):

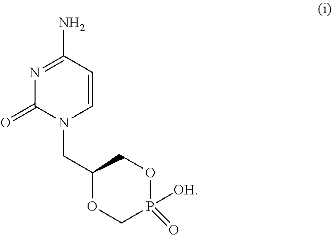

In further particular embodiments, the pharmaceutical, feed or water composition comprises an acyloxyalkyl ester, alkoxycarbonyloxyalkyl ester, alkoxyalkyl ester, or a phosphoramidate or phosphonamidite thereof.

In particular embodiments, the compositions described above comprise one or more compounds selected from the group consisting of:

| ID | Structure |
|---|---|
| cHPMPC | 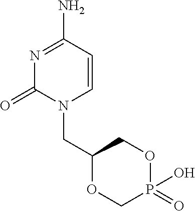 |
| 1 | 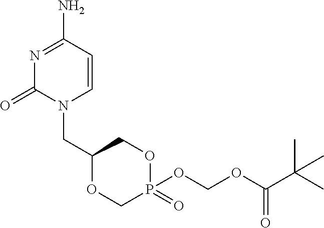 |
| 2 | 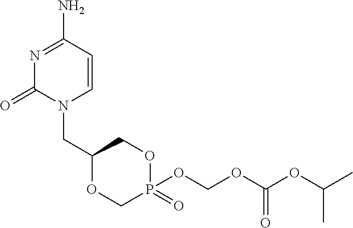 |
| 3 | 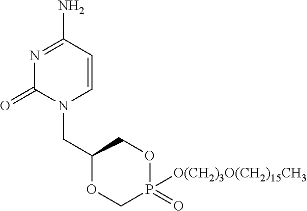 |
| 4 | 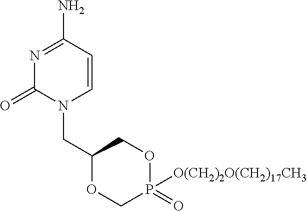 |
| 5 | 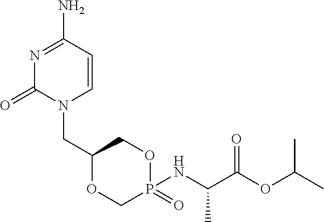 |

In further particular embodiments, the compounds are selected from the group consisting of compounds 2, 3, 4 and 5 above. The above compositions were found to be particularly suitable for use in the treatment of animals, most particularly for the treatment of viremia in animals. Particularly, these compositions were found to be useful for the treatment of African Swine Fever (ASF) in an animal.

The following examples are meant to illustrate the present invention and should not be construed as a limitation of its scope.

### EXAMPLES

### Example 1. Synthesis of cHPMPC

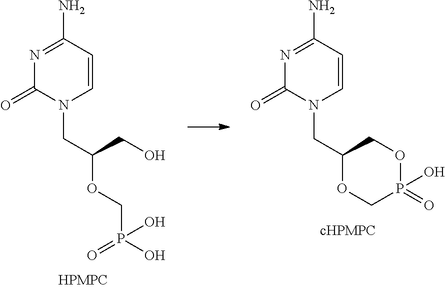

To a solution of (S)-2-(4-Amino-2-oxo-2H-pyrimidin-1-yl)-1-hydroxymethyl-ethoxymethyl]-phosphonic acid (140 g, 0.5 mol, 1.0 equiv) in Dimethyl formamide (140 mL, 10 V) was added N,N′-Dicyclohexyl-4-morpholinecarboxamidine (162 g, 1.1 equiv) and N,N-Dicyclohexylcarbodiimide (311 g, 3.0 equiv) at 25±5° C. The resulting reaction mass was heated to 90±5° C. and stirred for 8 hours at 90±5° C. After completion, reaction mass was cooled to room temperature and volatiles were evaporated under reduced pressure. The crude product was diluted with water (14 V) and filtered.

The filtrate was concentrated under reduced pressure. The crude product was dissolved in minimum amount of water and loaded on to a column of DOWEX 1*2 acetate form (20 w/w). Elution was performed by using increasing percentage of 1.0 M acetic acid in purified water. The desired product was collected at 3 to 7% of 1.0 M acetic acid in purified water. Product containing fractions were concentrated to obtain the product as off-white solid. Yield: 93.3 g, 71%.

### Example 2. Antiviral activity of cHPMPC against different ASFV strains in vitro

Primary peripheral blood mononuclear cells (PBMCs) treated with serial dilutions of cyclic cidofovir and infected with Georgia 2007/1, KAB6/2, BOT 1/99 or Magadi w/hog 9 at 0.1 MOI for 72 h. Progeny virus output (%) determined by hemadsorption assay. EC₅₀ values calculated by non-linear regression, mean EC₅₀ from three independent experiments stated with standard deviation.

TABLE 1

| EC₅₀ (μM) | EC₅₀ (μM) | EC₅₀ (μM) | EC₅₀ (μM) |
|---|---|---|---|
| Georgia 2007/1 | KAB6/2 | BOT P1/99 | Magadi w/hog 9 |
| 0.31 ± 0.17 | 0.59 ± 0.08 | 0.3 ± 0.2 | 0.11 ± 0.05 |

### Example 3. Cell toxicity

Purified peripheral blood mononuclear cells (PBMCs) treated with serial dilutions of cyclic cidofovir for 72 h and cell viability measured by RealTime-Glo™ MT Cell Viability Assay or CellTiter 96 Non-Radioactive Cell Proliferation Assay (MTT). CC₅₀ value (50% cytotoxic concentration) was calculated from relative cell viability (%) by non-linear regression. Four independent experiments performed in triplicate, values represent mean and standard deviation.

TABLE 2

| CC₅₀ (μM) | CC₅₀ (μM) |
|---|---|
| RealTime-Glo Assay | CellTiter 96 Assay |
| 39.43 ± 12.29 | 44.29 ± 4.74 |

### Example 4. Single-dose pharmacokinetic study in pigs

### Material and methods

### Test Material

cHPMPC 20 mg/mL aqueous solution (pH neutralized by NaHCO₃), also referred to herein as “VV03-0035”.

### Summary of Study Design

A total of three weaned piglets of three weeks and five days of age were sourced from a commercial pig unit. The animals were examined on arrival to confirm that they were in good health and were then ear tagged. The animals were housed as one group on straw. The animals were allowed to acclimatise for a period of 7 days before the onset of procedures.

On Day 0, once confirmed as being suitable for inclusion on study, all animals were weighed and blood sampled then each was administered the Test Material by the intravenous route. Post administration, blood samples were collected 10 min (±2 min), 30 min, (±2 min), 45 min (±2 min), 1 h (±2 min), 2 h (±2 min), 4 h (±2 min), 6 h (±2 min), 8h (±2 min), 12 h (±2 min) and 24 h (±2 min).

Post final blood sample the animals were allowed to recover for seven days. On Day 8 all animals were weighed, blood sampled then each was administered the Test Material by the oral route. Post administration, blood samples were collected 30 min (±2 min), 1 h (±2 min), 2 h (±2 min), 3 h (±2 min), 4 h (±2 min), 5 h (±2 min), 6 h (±2 min), 8 h (±2 min), 12 h (±2 min) and 24 h (±2 min).

Clinical observations including where relevant injection sites assessments were carried out on approximately 1 and 4 hours post administration on Day 0 and Day 8.

The study completed on Day 9. A summary of the study design is provided in Table 3 below.

TABLE 3
Summary of Study Design

| | No. of | Test | | | | End of |
|---|---|---|---|---|---|---|
| Group | animals | Material | Day | Route/Volume | Dose | Study |
| 1 | 3 | Test | 0 | IV/0.25 ml/kg | IV: 5 mg/kg | Day 9 |
| | | Material | 8 | Oral/1.25 ml/kg | Oral: 25 mg/kg | |

### Results

In this study VV03-0035 was well tolerated in pigs. Generally, the pigs did not show abnormal behavior or signs of injury or illness during the study and all pigs were considered to be in good general health during the observations carried out 1 and 4 hours post administration of VV03-0035. A redness at injection site was observed in all pigs 1 hour post intravenous dosing of VV03-0035. However, no injection site reactions were observed at the 4 hour post intravenous administration observation. No other adverse events were recorded following administration of VV03-0035 at 5 mg/kg intravenously or at 25 mg/kg orally.

The results obtained in this study showed that after a single intravenous administration of VV03-0035 at a dose of 5 mg/kg, or after a single oral administration of VV03-0035 at a dose of 25 mg/kg, all animals exhibited a measurable concentration of both VV03-0035 and cidofovir in plasma until 24 hours post administration (last sampling time) except for one pig (pig #342008) at 24 hours post oral dosing. This could be explained by the fact that pig #342008 did not receive the entire target dose of 25 mg/kg of VV03-0035, but an actual dose around 21.7 mg/kg on the day of oral administration, which led to a lower dose around 83100 nmol/kg compared to the 2 other animals with doses of 93900 and 92400 nmol/kg.

The mean maximum plasma concentration (Cₘₐₓ) after intravenous administration of VV03-0035 was 49504±18300 nM, and 2036±1340 nM after oral administration of VV03-0035. The latter was reached 2 hours after oral administration. The mean Cₘₐₓ of cidofovir after intravenous administration of VV03-0035 was 1816±86 nM and was reached 2 hours post administration for all 3 pigs. After oral administration of VV03-0035, the cidofovir Cₘₐₓ was 221±140 nM and was reached between 3 and 8 hours depending on the pigs. For VV03-0035, exposure (expressed as area under the curve or AUC) ranged from 58000 to 95400 nM.h with a mean at 71571 nM.h and inter-subject variability (% CV) of 29% for intravenous administration, while for the oral administration, exposure ranged from 2320 to 23700 nM.h with a mean at 11573 nM.h and inter-subject variability of 91% CV. Exposure parameters for cidofovir were less accurate due to the absence of apparent terminal elimination phase. Mean exposure for cidofovir after intravenous administration of VV03-0035 was 12044 nM.h and 3562 nM.h after oral administration of VV03-0035. Mean half-life (t_{1/2}) values for VV03-0035 were 3.31 hours and 3.51 hours after intravenous and oral administration, respectively. Mean t_{1/2} values for cidofovir were 9.37 hours and 11.23 hours after intravenous and oral administration of VV03-0035, respectively.

TABLE 4

| | | Analyte | | | Analyte | | |
|---|---|---|---|---|---|---|---|
| | Nominal | VV03-0035 | | | Cidofovir | | |
| | Dose | Cₘₐₓ | Tₘₐₓ | AUC | Cₘₐₓ | Tₘₐₓ | AUC |
| Route | (mg/kg) | (nM) | (h) | (nM · h) | (nM) | (h) | (nM · h) |
| IV | 5 | 49504 ± 18300 | 0.17 | 71571 ± 18300 | 1816 ± 86 | 2 | 12044 ± 2320 |
| PO | 25 | 2036 ± 1340 | 2 | 11573 ± 10400 | 221 ± 140 | 5 | 3562 ± 3090 |

### Example 5. VV03-0035 challenge study in pigs

### Material and methods

VV03-0035 was formulated as a 20 mg/ml aqueous solution (pH neutralized by NaHCO₃).

ASFV strain Georgia 2007/1 is a genotype II field isolate from the ASFV outbreak in Georgia in 2007 and is part of the ASFV strain collection available at the Pirbright Institute where the study was conducted.

### Study design

The two groups, group 1, the VV03-0035 treatment group (n=6 pigs, 3 males and 3 females) and group 2, a placebo group (n=3 mixed sex) were housed in one room. The pigs were assigned to treatment group according to a randomized block design. Female piglets (n=5) and male piglets (n=4) were sorted by weight from highest to lowest and assigned a random number generated by commercially available software (Excel). Starting with the lightest pair, the animal with the higher assigned random number was assigned to the VV03-0035 treatment group (Group 1) and the animal with the lower random number was assigned to the control group (Group 2).

Animals were allowed to settle in for 7 days and daily clinical scoring, including body temperatures started from 3 days before the start of the experiment until the end of the experiment. On Day -1 EDTA blood (1 ml) and serum (4 ml) was collected from each pig. Nasal, oral and rectal swabs were also collected from each pig.

Next, VV03-0035 or placebo (saline) was delivered orally twice daily (30 mg/kg, 1.5 mL/kg, 12-h intervals) for 3 days from day -1 to +1 after ASFV challenge (challenge was done on day 0). Between days 2 and 6 the drug was delivered once daily in the morning at approximately the same time each day. Pigs were kept for a further 21 days to observe clinical signs typical of ASFV or the drug treatment.

The VV03-0035 volume per pig was calculated based on daily body weights. Control pigs were given a placebo of the same volume per kg.

On Day 0, 30-60 minutes after the VV03-0035 or placebo administration to group 1 and group 2, respectively, all pigs were challenged with 10³ HAD₅₀ (50% haemadsorping dose) Georgia 2007/1 in 1 ml volume by the intramuscular route.

It was expected the Group 2 control pigs were to be culled by day 5 or 6 on humane welfare grounds.

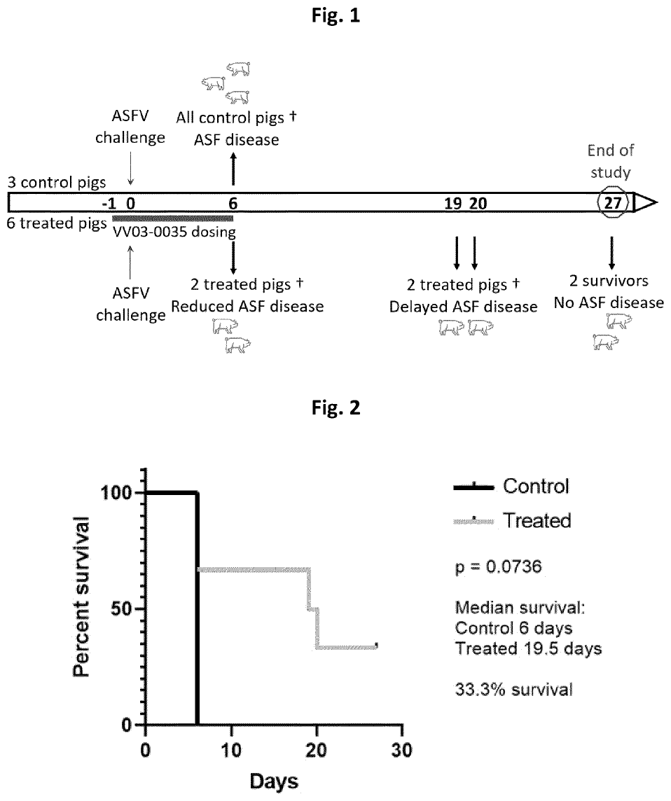

TABLE 5

| | No. of | Test | | | | End of |
|---|---|---|---|---|---|---|
| Group | pigs | material | Day | Route | Dose/Volume | dosing |
| 1 | 6 | VV03- | Twice daily for 3 days (from 1 | PO | 30 mg/kg | Day 6 post |
| | | 0035 | day before till 1 day after | | (1.5 ml/kg) | challenge |
| 2 | 3 | Saline | challenge) and once daily on | | 1.5 ml/kg | Day 6 post |
| | | | days 2 to 6 post challenge (see | | | challenge |
| | | | FIG. 1) | | | |

ASFV DNA was measured in whole blood by quantitative qPCR using the method described by King et al., Protection of European domestic pigs from virulent African isolates of African swine fever by experimental immunization, Vaccine 29 (2011):4593-4600 (further referred to herein as “King et al. (2011)”) using primers specific for the B646L ASFV gene encoding the VP72 protein. Results were expressed as genome copies per ml of blood by comparison with a standard curve of a control plasmid.

ASFV DNA was measured in in nasal, oral and rectal swabs by quantitative qPCR using the method described by King et al. (2011) using primers specific for the B646L ASFV gene encoding the VP72 protein. Virus was eluted from the swab samples in phosphate buffered saline. Results were expressed as genome copies per ml by comparison with a standard curve of a control plasmid.

### Results

All group 2 control pigs (n=3) were humanely euthanised on day 6 as a result of reaching a moderate severity humane endpoint due to ASF disease.

Of the six VV03-0035-treated pigs, 2 pigs were euthanized at a moderate severity humane endpoint on day 6 after ASFV challenge showing reduced ASF disease, 2 pigs were euthanized at a moderate severity humane endpoint on day 19 and day 20 after ASFV challenge with delayed and reduced ASF disease and the remaining 2 pigs survived until the end of the study (day 27 post ASFV challenge) with no ASF disease (FIGS. 1 and 2).

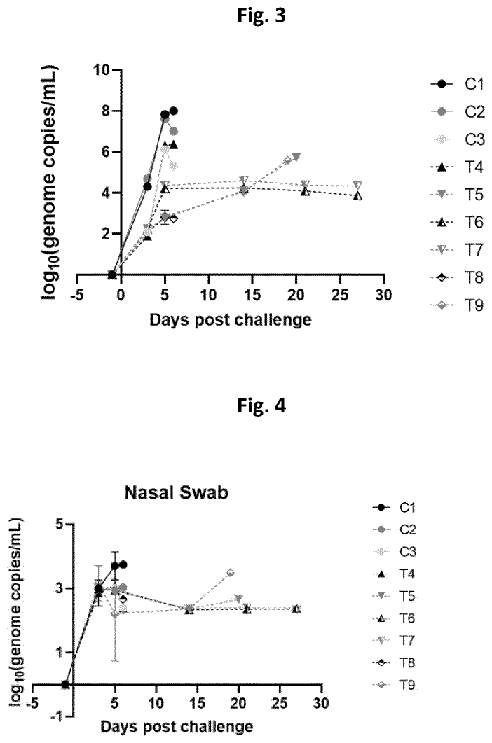

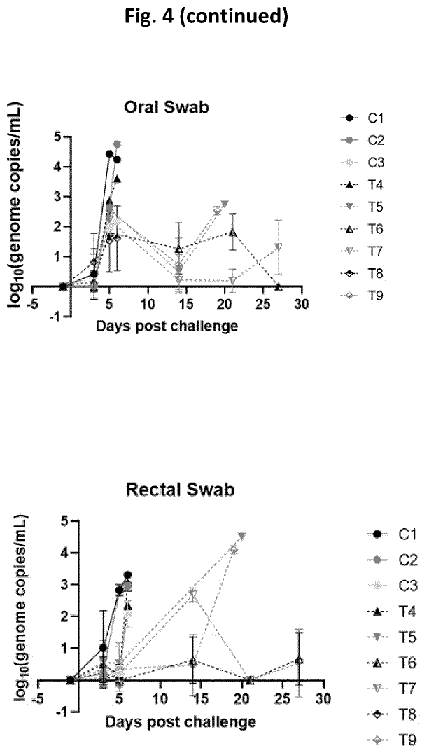

Present inventors found reduced ASFV genome copies in blood (FIG. 3) and swab samples of treated pigs on termination days compared to control pigs (FIG. 4).

### Example 6. VV03-0035 challenge study in pigs

### Material and methods

VV03-0035 was formulated as a 20 mg/ml aqueous solution (pH neutralized by NaHCO₃).

ASFV strain Georgia 2007/1 is a genotype II field isolate from the ASFV outbreak in Georgia and is part of the ASFV strain collection available at the Pirbright Institute where the study was conducted.

Study design

The two groups, group 1, the VV03-0035 treatment group (n=6 pigs, 3 males and 3 females) and group 2, a placebo group (n=6 pigs, 3 males and 3 females) were housed in two separate room. The pigs were assigned to treatment group according to a randomized block design. Female piglets (n=6) and male piglets (n=6) were sorted by weight from highest to lowest and assigned a random number generated by commercially available software (Excel). Starting with the lightest pair of the same gender, the animal with the higher assigned random number was assigned to the VV03-0035 treatment group (Group 1) and the animal with the lower random number was assigned to the control group (Group 2).

Animals were allowed to settle in for 7 days and daily clinical scoring, including temperatures started from 3 days before the start of the experiment until the end of the experiment. On Day -1 EDTA blood (1 ml) and serum (4 ml) was collected from each pig. Nasal, pharyngeal and rectal swabs were also collected from each pig.

Next, VV03-0035 or placebo (saline) was delivered orally (30 mg/kg, 1.5 mL/kg) once daily on days -1 (in the evening), day 0 (30-60 min before ASFV challenge), day 1 (in the morning) and day 2 (in the morning). Between days 2 and up to day 8 the drug or placebo was delivered every other day (on days 4 and 6) in the morning at approximately the same time each day. Feed was given after administration. The VV03-0035 volume per pig was calculated based on daily body weights. Control pigs were given a placebo of the same volume per kg.

On Day 0, 30-60 minutes after the VV03-0035 or placebo administration to group 1 and group 2, respectively, all pigs were challenged with 10³ HAD₅₀ (50% haemadsorping dose) Georgia 2007/1 in 1 ml volume by the intramuscular route.

It was expected the Group 2 control pigs were to be culled between day 4 and 6 on humane welfare grounds at a moderate severity humane endpoint.

Tissue samples including spleen, lung, sub-mandibular (SMLN), renal (RNL) and gastro-hepatic lymph (GHLN) nodes were collected at necropsy and stored at −80° C. until processed. Nucelic acid was extracted from 20 mg of homogenised tissue and the ASFV genome copies determined by quantitative qPCR using primers specific for B646L gene (King et al., 2011) in comparison with a standard control plasmid.

Blood samples in EDTA were collected at different time points pre- or post-challenge with ASFV from Group 1 and Group 2 pigs. ASFV genome copies per ml of blood were measured by quantitative qPCR (King et al., 2011) using primers specific for the B646L ASFV gene and by comparison with a standard curve of a control plasmid.

TABLE 6

| | No. of | Test | | | | End of |
|---|---|---|---|---|---|---|
| Group | pigs | material | Day | Route | Dose/Volume | dosing |
| 1 | 6 | VV03- | Once daily on days −1, 0, | PO | 30 mg/kg | Day 6 post |
| | | 0035 | 1, 2, 4, 6, post challenge | | (1.5 ml/kg) | challenge |
| 2 | 6 | Saline | | | 1.5 ml/kg | Day 6 post |
| | | | | | | challenge |

### Results

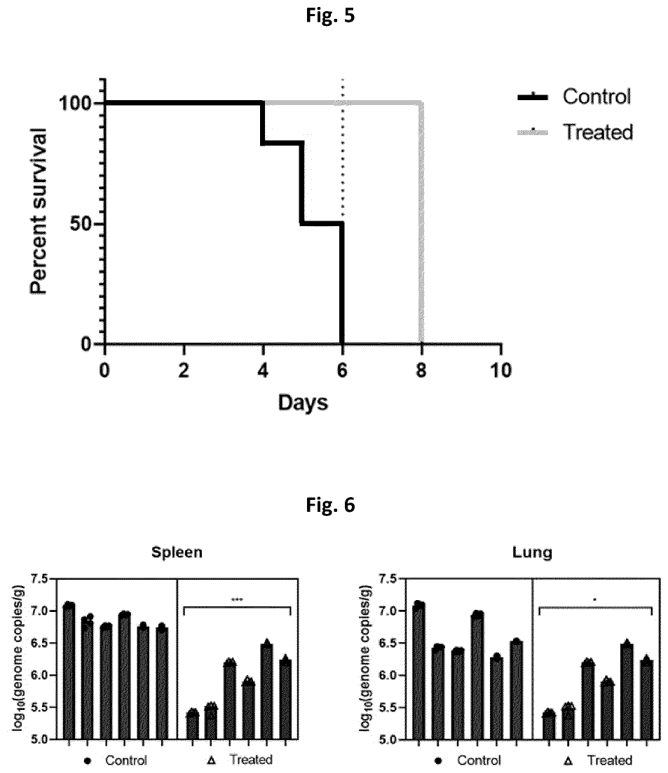

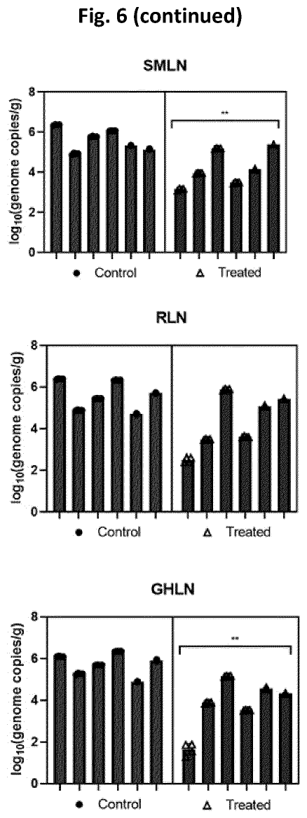

VV03-0035 improved survival time in pigs challenged with Georgia 2007/1. A significant difference in median day of death was observed with VV03-0035 treated pigs surviving until day 8 whereas the median day of death equaled 5.5 days for the negative control animals. This difference was statistically significant as determined by log-rank (Mantel-Cox) test and Gehan-Breslow-Wilcoxon test with p-values of 0.0010 and 0.0016, respectively. (FIG. 5) Compared to the placebo group, VV03-0035 significantly reduced ASF viral genome copies in several organs such as lungs, spleens and several lymph nodes as measured by quantifying viral genomes in tissue using quantitative qPCR. Significance was calculated by nested t comparing control pigs to treated pigs (FIG. 6).

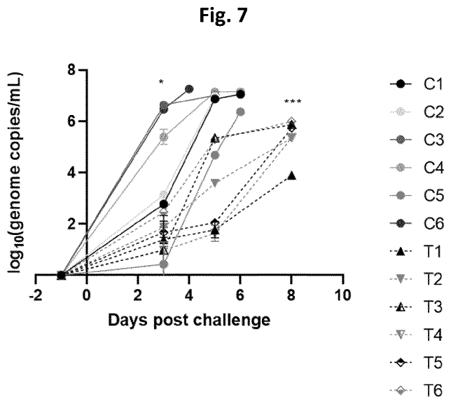

VV03-0035 significantly reduced ASF viraemia as measured by quantitative qPCR. Significance was calculated by nested t comparing control pigs to treated pigs within each timepoint (FIG. 7).

### Example 7. Synthesis of cHMPC derivatives

### Pivaloyloxymethyl ester of cHPMPC (POM-cHPMPC, Compound 1)

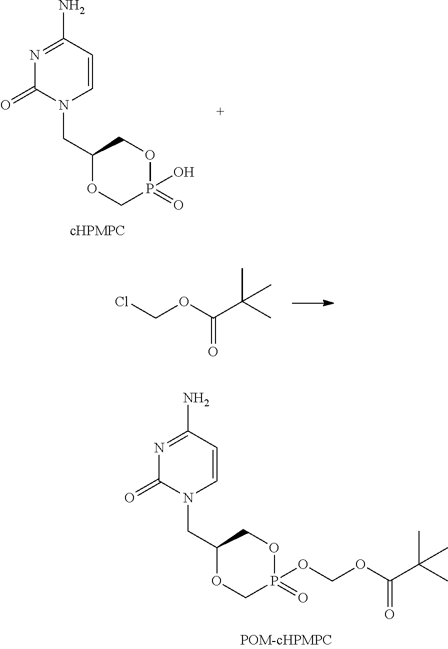

Tetrabutylammonium hydroxide (0.8 mL; 25% in methanol) was added dropwise to a stirred mixture of cHPMPC (200 mg; 0.766 mmol) in methanol (24 mL) under inert atmosphere. The mixture was stirred for 10 min till solubilisation. Then the solvent was evaporated, and the remaining methanol was stripped with dioxane (2×6 mL). The residue was dissolved in dioxane (10 mL) and chloromethyl pivalate (0.13 mL; 1.2 eq.) was added. The mixture was stirred at 90° C. for 1 h. The reaction was monitored by TLC. After cooling to room temperature, the volatiles were evaporated. The residue was chromatographed on silica gel column, further purified by prep HPLC, and lyophilized to obtain the product as a white solid (30 mg; 11% yield) and as a mixture of diastereomers ˜5:2. ¹H NMR (400 MHz, D₂O) δ ppm: 7.51 and 7.45 (d, 1H); 5.98-5.85 (m, 1H); 5.78-5.55 (m, 2H); 4.60-3.70 (m, 7H); 1.16 and 1.15 (s, 9H). ES+MS m/z: 376.1 (M+1).

### Isopropyloxycarbonyloxymethyl ester of cHPMPC (POC-cHPMPC, Compound 2)

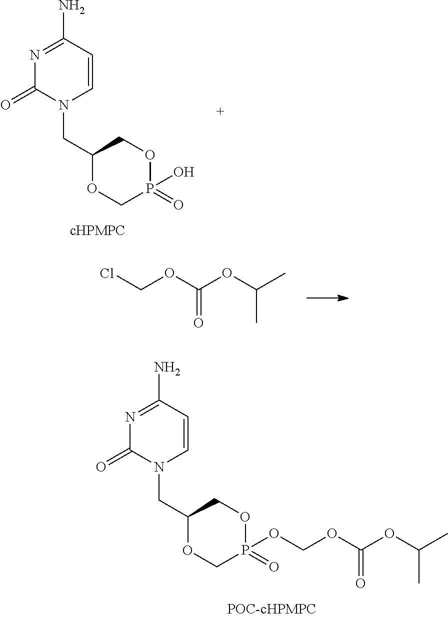

Tetrabutylammonium hydroxide (0.8 mL; 25% in methanol) was added dropwise to a stirred mixture of cHPMPC (200 mg; 0.766 mmol) in methanol (24 mL) under inert atmosphere. The mixture was stirred for 10 min till solubilisation. Then the solvent was evaporated, and the remaining methanol was stripped with dioxane (2×6 mL). The residue was dissolved in dioxane (10 mL) and chloromethyl isopropyl carbonate (0.14 mL; 1.2 eq.) was added. The mixture was stirred at 90° C. for 1 h. The reaction was monitored by TLC. After cooling to room temperature, the volatiles were evaporated. The residue was chromatographed on silica gel column, further purified by prep HPLC, and lyophilized to obtain the product as a white solid (30 mg; 11% yield) and as a mixture of diastereomers ˜1:1.2. ¹H NMR (400 MHz, D₂O) δ ppm: 7.53 and 7.47 (d, 1H); 6.00-5.85 (m, 1H); 5.80-5.60 (m, 2H); 4.98-4.85 (m, 1H); 4.60-3.68 (m, 7H); 1.28 and 1.26 (d, 6H). ES+MS m/z: 378.1 (M+1).

### Hexadecyloxypropyl ester of cHPMPC (HDP-cHPMPC, Compound 3)

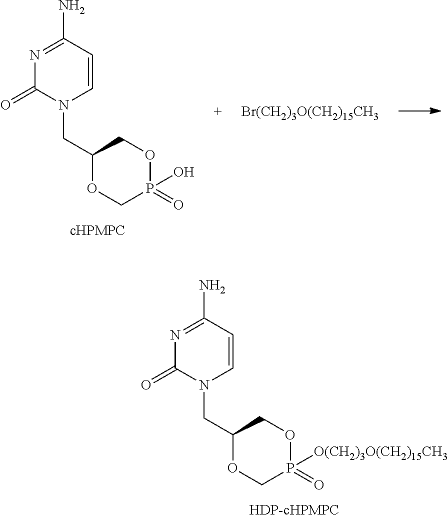

A mixture of cHPMPC (200 mg; 0.766 mmol) and N,N′-Dicyclohexylmorpholine-4-carboxaminine (240 mg; 1.1eq.) in DMF (2 mL) was stirred under inert atmosphere at room temperature for 12h till complete solubilisation. Then 1-bromo-3-hexadecyloxypropane (270 mg, 1 eq.) was added, the mixture was heated to 80° C. and stirred for 6 h. The reaction was monitored by TCL. After cooling to room temperature, the mixture was concentrated in vacuo. The residue was chromatographed on silica gel column, further purified by prep HPLC, and lyophilized to obtain the product as a white solid (35 mg; 9% yield) and as a mixture of diastereomers ˜1:1.3.1H NMR (400 MHz, CDCl₃) δ ppm: 7.29 and 7.27 (d, 1H); 5.67 (dd, 1H); 4.48-4.32 (m, 1H); 4.30-4.00 (m, 5H); 3.95-3.34 (m, 5H); 2.05-1.48 (m, 5H); 1.25 (broad s, 27H); 0.88 (t, 3H). ES+MS m/z: 544.5 (M+1).

### Octadecyloxyethyl ester of cHPMPC (ODE-cHPMPC, Compound 4)

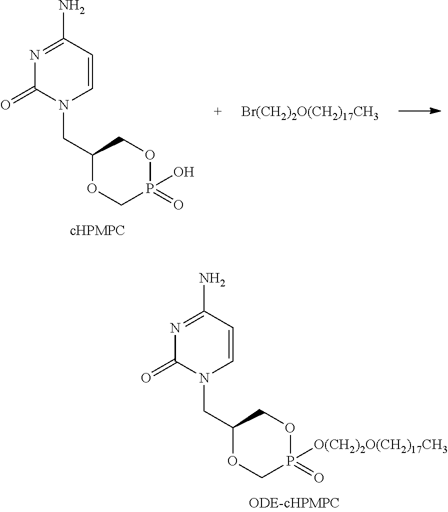

A mixture of cHPMPC (200 mg; 0.766 mmol) and N,N′-Dicyclohexylmorpholine-4-carboxaminine (240 mg; 1.1eq.) in DMF (5 mL) was stirred under inert atmosphere at room temperature for 12 h. Then 1-bromo-2-octadecyloxyethane (289 mg, 1eq.) was added, the mixture was heated to 80° C. and stirred for 6 h. After cooling to room temperature, the mixture was concentrated in vacuo. The residue was chromatographed on silica gel column, further purified by prep HPLC, and lyophilized to obtain the product as a white solid (42 mg; 10% yield) and as a mixture of diastereomers ˜1:1.7. ¹H NMR (400 MHz, CDCl₃) δ ppm: 7.32 and 7.30 (d, 1H); 5.90-5.78 (m, 1H); 4.48-4.03 (m, 7H); 3.94-3.82 (m, 1H); 3.74-3.38 (m, 5H); 1.70-1.50 (m, 4H); 1.25 (broad s, 28H); 0.88 (t, 3H). ES+MS m/z: 558.5 (M+1).

### Isopropyl L-alaninyl cHPMPC (Compound 5)

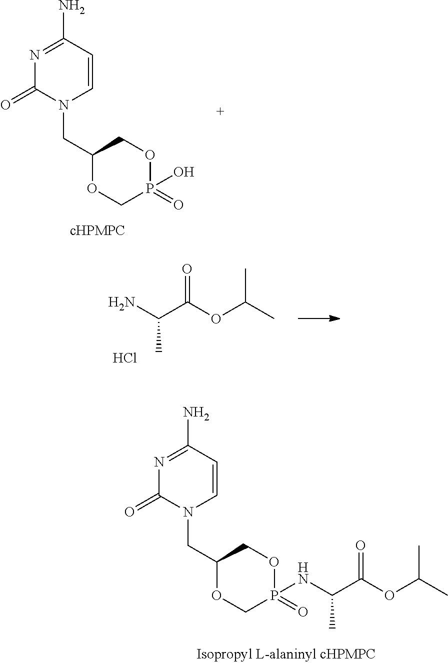

cHPMPC (200 mg; 0.766 mmol) was dissolved in DMF (1 mL) under inert atmosphere. Benzotriazol-1-yloxytris(dimethylamino)phosphonium hexafluorophosphate (500 mg; 1.5 eq.), N,N-diisopropylethylamine (0.3 mL; 2 eq.) and isopropyl L-alaninate hydrochloride (120 mg; 1 eq.) was added to the stirred solution. The reaction mixture was stirred for 3 hours at 55° C. The reaction was monitored by TLC. After cooling to room temperature, the mixture was concentrated in vacuo. The residue was chromatographed on silica gel column, further purified by prep HPLC, and lyophilized to obtain the product as a white solid (5 mg; 2% yield) and as a mixture of diastereomers ˜1:1. ¹H NMR (400 MHz, D₂O) δ ppm: 7.52 (d, 1H), 5.94 and 5.92 (d, 1H); 5.05-4.90 (m, 1H); 4.50-3.60 (m, 8H); 1.37 and 1.32 (d, 3H); 1.25-1.12 (m, 6H). ES+MS m/z: 375.1 (M+1).

### Example 8. Trial of Compounds 1 to 5 compared to cHPMPC

### Method

Compounds 1 to 5 and cHPMPC were diluted in PBS and added to porcine bone marrow cells cultured in EBSS supplemented with 10% porcine serum and 1% penicillin-streptomycin in triplicate wells of a 96 well plate at final concentrations of, A: 1.0 μM, B: 0.5 μM, C: 0.1 μM, D: 0.05 μM, E: 0.01 μM, F: 0.005 μM, G 0.001 μM.

The cells were infected at a low multiplicity (0.1 MOI) with a recombinant ASFV genotype II Georgia virus, GeorgiaDMGFB, expressing mNeon Green fluorescent protein under control of the ASFV early promoter p30. This reporter gene was inserted in place of four non-essential genes belonging to multigene families MGF360 and MGF 505 (https://iournals.asm.org/doi/10.1128/ivi.01899-21) and did not cause a reduction in virus replication or virulence. As controls infected cells without drug treatment were included.

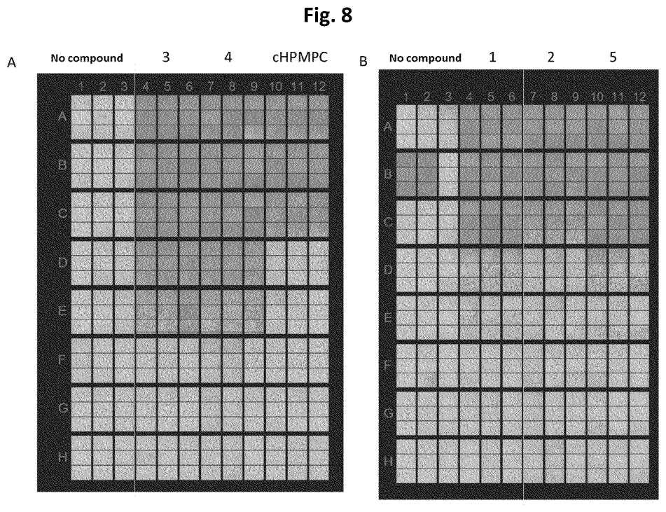

At 72 hours post-infection cells were visualised using an IncuCyte S3 (Sartorius) to measure expression of the mNeon Green Fluorescent protein as an indicator of virus infection. As shown in FIGS. 8A and 8B variation was observed in mNeon Green expression at different drug concentrations varying between different drugs used for treatment. Of particular interest mNeon Green expression was not observed in cells treated with Compounds 3 and 4 above a concentration of 0.005 μM (FIG. 8A Row F lanes 4, 5, 6 (Compound 3) and 7,8, 9 (Compound 4)) compared to above concentrations of 0.05 μM for compound cHPMPC (FIG. 8A Row D lanes 10, 11, 12). These results indicate a greatly increased efficacy of Compounds 3 and 4 compared to cHPMPC. Fluorescence detected in cells infected in the presence of Compounds 1, 2 and 5 (FIG. 8B) indicate that these compounds had efficacy intermediate between Compounds 3 and 4 and that of cHPMPC.

Compounds 3 and 4 are of special interest and have similar or lower CC₅₀ values (50% cytotoxic concentration) as cHPMPC (47+/−13 or 59+/−19 μM in porcine kidney cells). This dramatically increases selectivity indices to a range in thousands similar to that reported by (Lipka et al., 2023). Thus it can be expected that the prepared compounds would be effective at a much lower dose.

1-11. (canceled)

12. A method of treating an animal suffering from African Swine Fever (ASF), said method comprising orally administering to said animal at least one dose of from 10.0 to 30.0 mg/kg of cyclic cidofovir, wherein
in a first period of from 1 to 4 consecutive days said at least one dose of cyclic cidofovir is administered daily and
in a second period from 5 to 14 consecutive days said at least one dose of cyclic cidofovir is administered to the animal every other day or daily, and
wherein the average daily dose administered in said first period is higher than the average daily dose administered in said second period.

13. The method according to claim 12, wherein the cyclic cidofovir is a compound of formula (i): 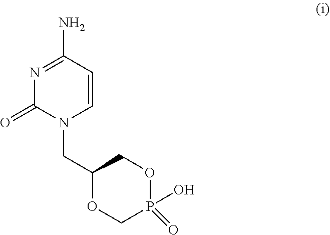
or an ester, an amidate or esteramidate thereof, more particularly an acyloxyalkyl ester, alkoxycarbonyloxyalkyl ester, alkoxyalkyl ester, or a phosphoramidate or phosphonamidite thereof.

14. The method according to claim 13, wherein the cyclic cidofovir has a structure of formula (I) or (II), 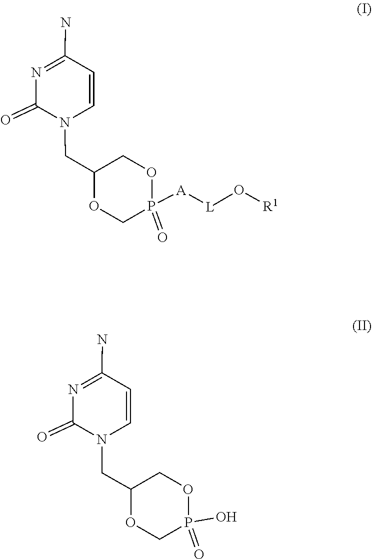
wherein,
A is selected from the group consisting of O and NH;
L is selected from the group consisting of —(CR²R³)ₙ—, —CH(R⁴)—C(O)— and —C(O)—CH(R⁵)—;
n is an integer selected from 1, 2, 3, 4 or 5;
R¹ is selected from the group consisting of C₆₋₂₅alkyl, —C(O)R⁶, —CO₂R⁷, C₆₋₂₅haloalkyl, C₆₋₂₅alkenyl, C₆₋₂₅haloalkenyl, C₃₋₁₂cycloalkyl and C₃₋₁₂halocycloalkyl;
each R² is independently selected from the group consisting of hydrogen, C₁₋₆alkyl, C₃₋₁₂cycloalkyl and halogen;
each R³ is independently selected from the group consisting of hydrogen, C₁₋₆alkyl, C₃₋₁₂cycloalkyl and halogen;
each R⁴ is independently selected from the group consisting of hydrogen, C₁₋₆alkyl, C₃₋₁₂cycloalkyl and halogen;
each R⁵ is independently selected from the group consisting of hydrogen, C₁₋₆alkyl, C₃₋₁₂cycloalkyl and halogen;
each R⁶ is independently selected from the group consisting of C₁₋₁₀alkyl, C₁₋₁₀haloalkyl, C₃₋₁₂cycloalkyl, C₃₋₁₂halocycloalkyl, C₂₋₁₀alkeny, and C₂₋₁₀haloalkenyl;
each R⁷ is independently selected from the group consisting of C₁₋₁₀alkyl, C₁₋₁₂haloalkyl, C₃₋₁₂cycloalkyl, C₃₋₁₂halocycloalkyl, C₂₋₁₀alkeny, and C₂₋₁₀haloalkenyl;
or a stereoisomer, tautomer, solvate, hydrate, pharmaceutically acceptable salt thereof.

15. The method according to claim 14, wherein
L is selected from the group consisting of —(CR²R³)ₙ— and —CH(R⁴)—C(O)—;
n is an integer selected from 1, 2, or 3;
R¹ is selected from the group consisting of C₆₋₂₅alkyl, —C(O)R⁶, —CO₂R⁷, C₆₋₂₅haloalkyl, C₆₋₂₅alkenyl, and C₃₋₁₀cycloalkyl;
each R² is independently selected from the group consisting of hydrogen, C₁₋₆alkyl, and halogen;
each R³ is independently selected from the group consisting of hydrogen, C₁₋₆alkyl, and halogen;
each R⁴ is independently selected from the group consisting of hydrogen, C₁₋₆alkyl, and halogen;
each R⁵ is independently selected from the group consisting of hydrogen, C₁₋₆alkyl, C₃₋₁₀cycloalkyl and halogen;
each R⁶ is independently selected from the group consisting of C₁₋₆alkyl, C₁₋₆haloalkyl, and C₃₋₆cycloalkyl;
each R⁷ is independently selected from the group consisting of C₁₋₆alkyl, C₁₋₆haloalkyl, and C₃₋₆cycloalkyl.

16. The method according to claim 15, selected from the group consisting of:

| ID | Structure |
|---|---|
| cHPMPC | 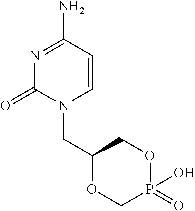 |
| 1 | 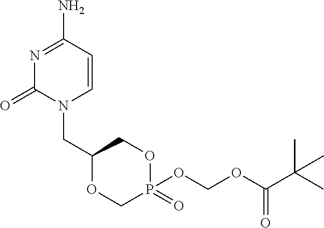 |
| 2 | 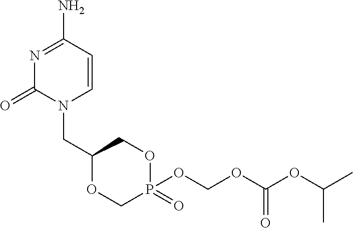 |
| 3 | 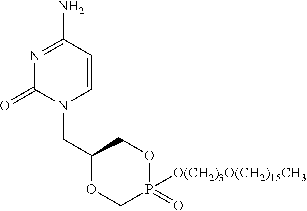 |
| 4 | 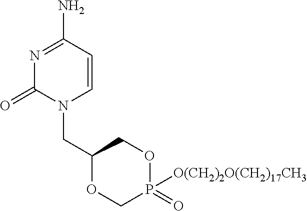 |
| 5 | 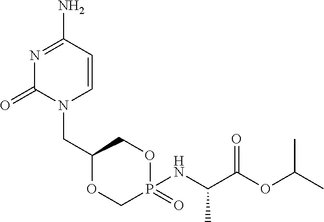 |

17. The method according to claim 12, wherein two doses of cyclic cidofovir are administered to the animal daily for a first period of 3 consecutive days and one dose of cyclic cidofovir is administered to the animal daily for a second period of from 5 to 14 consecutive days, preferably from 5 to 7 consecutive days.

18. The method according to claim 12, wherein one dose of cyclic cidofovir is administered to the animal daily for a first period of 3 days and one dose of cyclic cidofovir is administered to the animal every other day for a second period of from 5 to 14 consecutive days, preferably from 5 to 7 consecutive days.

19. The method according to claim 12, wherein the cyclic cidofovir is comprised in a pharmaceutical composition, preferably wherein said pharmaceutical composition is an aqueous solution.

20. The method according to claim 12, wherein the animal is a pig.

21. The method according to claim 12, wherein the animal is housed in an ASF surveillance or protection zone.

22. The method according to claim 12, wherein the administration of said cyclic cidofovir prevents the spread of ASF in an animal population.
